SEQUENCE LISTING (49 sequences)

SEQ ID NO: 1 - RNA, 23 nt, synthetic construct
  source 1..23: mol_type = other RNA
acaaaagcaaaacaggtctagaa

SEQ ID NO: 2 - RNA, 21 nt, synthetic construct
  source 1..21: mol_type = other RNA
ctagacctgttttgcttttgt

SEQ ID NO: 3 - RNA, 21 nt, synthetic construct
  source 1..21: mol_type = other RNA
  modified_base 11: mod_base = OTHER; note = thymine
ctagacctgttttgcttttgt

SEQ ID NO: 4 - RNA, 21 nt, synthetic construct
  source 1..21: mol_type = other RNA
  modified_base 1^2: mod_base = OTHER; note = Phosphorothioate Linkage
  modified_base 2^3: mod_base = OTHER; note = Phosphorothioate Linkage
  modified_base 1: mod_base = OTHER; note = 2&apos;-fluoronucleotide
  modified_base 3: mod_base = OTHER; note = 2&apos;-fluoronucleotide
  modified_base 7: mod_base = OTHER; note = 2&apos;-fluoronucleotide
  modified_base 9..11: mod_base = OTHER; note = 2&apos;-fluoronucleotide
  modified_base 2: mod_base = OTHER; note = 2&apos;-O-methylnucleotide
  modified_base 4..6: mod_base = OTHER; note = 2&apos;-O-methylnucleotide
  modified_base 8: mod_base = OTHER; note = 2&apos;-O-methylnucleotide
  modified_base 12..21: mod_base = OTHER; note = 2&apos;-O-methylnucleotide
ctagacctgttttgcttttgt

SEQ ID NO: 5 - RNA, 21 nt, synthetic construct
  source 1..21: mol_type = other RNA
  modified_base 1^2: mod_base = OTHER; note = Phosphorothioate Linkage
  modified_base 2^3: mod_base = OTHER; note = Phosphorothioate Linkage
  modified_base 1: mod_base = OTHER; note = 2&apos;-fluoronucleotide
  modified_base 3: mod_base = OTHER; note = 2&apos;-fluoronucleotide
  modified_base 7: mod_base = OTHER; note = 2&apos;-fluoronucleotide
  modified_base 9..11: mod_base = OTHER; note = 2&apos;-fluoronucleotide
  modified_base 2: mod_base = OTHER; note = 2&apos;-O-methylnucleotide
  modified_base 4..6: mod_base = OTHER; note = 2&apos;-O-methylnucleotide
  modified_base 8: mod_base = OTHER; note = 2&apos;-O-methylnucleotide
  modified_base 15..21: mod_base = OTHER; note = 2&apos;-O-methylnucleotide
  modified_base 12..13: mod_base = OTHER; note = 2&apos;-O-methylnucleotide
  modified_base 14: mod_base = OTHER; note = 2&apos;-fluoronucleotide
ctagacctgttttgcttttgt

SEQ ID NO: 6 - RNA, 21 nt, synthetic construct
  source 1..21: mol_type = other RNA
  modified_base 1^2: mod_base = OTHER; note = Phosphorothioate Linkage
  modified_base 2^3: mod_base = OTHER; note = Phosphorothioate Linkage
  modified_base 1: mod_base = OTHER; note = 2&apos;-fluoronucleotide
  modified_base 3: mod_base = OTHER; note = 2&apos;-fluoronucleotide
  modified_base 7: mod_base = OTHER; note = 2&apos;-fluoronucleotide
  modified_base 9..11: mod_base = OTHER; note = 2&apos;-fluoronucleotide
  modified_base 2: mod_base = OTHER; note = 2&apos;-O-methylnucleotide
  modified_base 4..6: mod_base = OTHER; note = 2&apos;-O-methylnucleotide
  modified_base 8: mod_base = OTHER; note = 2&apos;-fluoronucleotide
  modified_base 15..21: mod_base = OTHER; note = 2&apos;-O-methylnucleotide
  modified_base 12..13: mod_base = OTHER; note = 2&apos;-O-methylnucleotide
  modified_base 14: mod_base = OTHER; note = 2&apos;-fluoronucleotide
ctagacctgttttgcttttgt

SEQ ID NO: 7 - RNA, 21 nt, synthetic construct
  source 1..21: mol_type = other RNA
  modified_base 1^2: mod_base = OTHER; note = Phosphorothioate Linkage
  modified_base 2^3: mod_base = OTHER; note = Phosphorothioate Linkage
  modified_base 1: mod_base = OTHER; note = 2&apos;-fluoronucleotide
  modified_base 3: mod_base = OTHER; note = 2&apos;-fluoronucleotide
  modified_base 7: mod_base = OTHER; note = 2&apos;-fluoronucleotide
  modified_base 9: mod_base = OTHER; note = 2&apos;-fluoronucleotide
  modified_base 2: mod_base = OTHER; note = 2&apos;-O-methylnucleotide
  modified_base 4..6: mod_base = OTHER; note = 2&apos;-O-methylnucleotide
  modified_base 8: mod_base = OTHER; note = 2&apos;-O-methylnucleotide
  modified_base 12..21: mod_base = OTHER; note = 2&apos;-O-methylnucleotide
  modified_base 11: mod_base = OTHER; note = 2&apos;-deoxythymidine
  modified_base 10: mod_base = OTHER; note = 2&apos;-O-methylnucleotide
ctagacctgttttgcttttgt

SEQ ID NO: 8 - RNA, 21 nt, synthetic construct
  source 1..21: mol_type = other RNA
  modified_base 1^2: mod_base = OTHER; note = Phosphorothioate Linkage
  modified_base 2^3: mod_base = OTHER; note = Phosphorothioate Linkage
  modified_base 1: mod_base = OTHER; note = 2&apos;-fluoronucleotide
  modified_base 3: mod_base = OTHER; note = 2&apos;-fluoronucleotide
  modified_base 7: mod_base = OTHER; note = 2&apos;-fluoronucleotide
  modified_base 9: mod_base = OTHER; note = 2&apos;-fluoronucleotide
  modified_base 2: mod_base = OTHER; note = 2&apos;-O-methylnucleotide
  modified_base 4..6: mod_base = OTHER; note = 2&apos;-O-methylnucleotide
  modified_base 8: mod_base = OTHER; note = 2&apos;-O-methylnucleotide
  modified_base 12..13: mod_base = OTHER; note = 2&apos;-O-methylnucleotide
  modified_base 11: mod_base = OTHER; note = 2&apos;-deoxythymidine
  modified_base 10: mod_base = OTHER; note = 2&apos;-O-methylnucleotide
  modified_base 14: mod_base = OTHER; note = 2&apos;-fluoronucleotide
  modified_base 15..21: mod_base = OTHER; note = 2&apos;-O-methylnucleotide
ctagacctgttttgcttttgt

SEQ ID NO: 9 - RNA, 21 nt, synthetic construct
  source 1..21: mol_type = other RNA
  modified_base 1^2: mod_base = OTHER; note = Phosphorothioate Linkage
  modified_base 2^3: mod_base = OTHER; note = Phosphorothioate Linkage
  modified_base 1: mod_base = OTHER; note = 2&apos;-fluoronucleotide
  modified_base 3: mod_base = OTHER; note = 2&apos;-fluoronucleotide
  modified_base 7: mod_base = OTHER; note = 2&apos;-fluoronucleotide
  modified_base 9: mod_base = OTHER; note = 2&apos;-fluoronucleotide
  modified_base 2: mod_base = OTHER; note = 2&apos;-O-methylnucleotide
  modified_base 4..6: mod_base = OTHER; note = 2&apos;-O-methylnucleotide
  modified_base 8: mod_base = OTHER; note = 2&apos;-fluoronucleotide
  modified_base 12..13: mod_base = OTHER; note = 2&apos;-O-methylnucleotide
  modified_base 11: mod_base = OTHER; note = 2&apos;-deoxythymidine
  modified_base 10: mod_base = OTHER; note = 2&apos;-O-methylnucleotide
  modified_base 14: mod_base = OTHER; note = 2&apos;-fluoronucleotide
  modified_base 15..21: mod_base = OTHER; note = 2&apos;-O-methylnucleotide
ctagacctgttttgcttttgt

SEQ ID NO: 10 - RNA, 21 nt, synthetic construct
  source 1..21: mol_type = other RNA
  modified_base 1^2: mod_base = OTHER; note = Phosphorothioate Linkage
  modified_base 2^3: mod_base = OTHER; note = Phosphorothioate Linkage
  modified_base 1: mod_base = OTHER; note = 2&apos;-fluoronucleotide
  modified_base 3: mod_base = OTHER; note = 2&apos;-fluoronucleotide
  modified_base 7: mod_base = OTHER; note = 2&apos;-fluoronucleotide
  modified_base 9: mod_base = OTHER; note = 2&apos;-fluoronucleotide
  modified_base 2: mod_base = OTHER; note = 2&apos;-O-methylnucleotide
  modified_base 4..6: mod_base = OTHER; note = 2&apos;-O-methylnucleotide
  modified_base 8: mod_base = OTHER; note = 2&apos;-O-methylnucleotide
  modified_base 12..13: mod_base = OTHER; note = 2&apos;-O-methylnucleotide
  modified_base 11: mod_base = OTHER; note = 2&apos;-deoxythymidine
  modified_base 10: mod_base = OTHER; note = 2&apos;-fluoronucleotide
  modified_base 14: mod_base = OTHER; note = 2&apos;-fluoronucleotide
  modified_base 15..21: mod_base = OTHER; note = 2&apos;-O-methylnucleotide
ctagacctgttttgcttttgt

SEQ ID NO: 11 - RNA, 23 nt, synthetic construct
  source 1..23: mol_type = other RNA
  modified_base 1^2: mod_base = OTHER; note = Phosphorothioate Linkage
  modified_base 2^3: mod_base = OTHER; note = Phosphorothioate Linkage
  modified_base 21^22: mod_base = OTHER; note = Phosphorothioate Linkage
  modified_base 22^23: mod_base = OTHER; note = Phosphorothioate Linkage
  modified_base 2: mod_base = OTHER; note = 2&apos;-fluoronucleotide
  modified_base 4..6: mod_base = OTHER; note = 2&apos;-fluoronucleotide
  modified_base 8: mod_base = OTHER; note = 2&apos;-fluoronucleotide
  modified_base 10: mod_base = OTHER; note = 2&apos;-fluoronucleotide
  modified_base 12: mod_base = OTHER; note = 2&apos;-fluoronucleotide
  modified_base 14: mod_base = OTHER; note = 2&apos;-fluoronucleotide
  modified_base 16: mod_base = OTHER; note = 2&apos;-fluoronucleotide
  modified_base 18: mod_base = OTHER; note = 2&apos;-fluoronucleotide
  modified_base 1: mod_base = OTHER; note = 2&apos;-O-methylnucleotide
  modified_base 3: mod_base = OTHER; note = 2&apos;-O-methylnucleotide
  modified_base 7: mod_base = OTHER; note = 2&apos;-O-methylnucleotide
  modified_base 9: mod_base = OTHER; note = 2&apos;-O-methylnucleotide
  modified_base 11: mod_base = OTHER; note = 2&apos;-O-methylnucleotide
  modified_base 13: mod_base = OTHER; note = 2&apos;-O-methylnucleotide
  modified_base 15: mod_base = OTHER; note = 2&apos;-O-methylnucleotide
  modified_base 17: mod_base = OTHER; note = 2&apos;-O-methylnucleotide
  modified_base 19..23: mod_base = OTHER; note = 2&apos;-O-methylnucleotide
acaaaagcaaaacaggtctagaa

SEQ ID NO: 12 - RNA, 23 nt, synthetic construct
  source 1..23: mol_type = other RNA
  modified_base 1^2: mod_base = OTHER; note = Phosphorothioate Linkage
  modified_base 2^3: mod_base = OTHER; note = Phosphorothioate Linkage
  modified_base 21^22: mod_base = OTHER; note = Phosphorothioate Linkage
  modified_base 22^23: mod_base = OTHER; note = Phosphorothioate Linkage
  modified_base 2: mod_base = OTHER; note = 2&apos;-fluoronucleotide
  modified_base 4..6: mod_base = OTHER; note = 2&apos;-fluoronucleotide
  modified_base 8: mod_base = OTHER; note = 2&apos;-fluoronucleotide
  modified_base 10: mod_base = OTHER; note = 2&apos;-fluoronucleotide
  modified_base 12: mod_base = OTHER; note = 2&apos;-fluoronucleotide
  modified_base 14: mod_base = OTHER; note = 2&apos;-fluoronucleotide
  modified_base 16: mod_base = OTHER; note = 2&apos;-fluoronucleotide
  modified_base 18: mod_base = OTHER; note = 2&apos;-fluoronucleotide
  modified_base 1: mod_base = OTHER; note = 2&apos;-fluoronucleotide
  modified_base 3: mod_base = OTHER; note = 2&apos;-O-methylnucleotide
  modified_base 7: mod_base = OTHER; note = 2&apos;-O-methylnucleotide
  modified_base 9: mod_base = OTHER; note = 2&apos;-O-methylnucleotide
  modified_base 11: mod_base = OTHER; note = 2&apos;-O-methylnucleotide
  modified_base 13: mod_base = OTHER; note = 2&apos;-O-methylnucleotide
  modified_base 15: mod_base = OTHER; note = 2&apos;-O-methylnucleotide
  modified_base 17: mod_base = OTHER; note = 2&apos;-O-methylnucleotide
  modified_base 19..23: mod_base = OTHER; note = 2&apos;-O-methylnucleotide
acaaaagcaaaacaggtctagaa

SEQ ID NO: 13 - RNA, 23 nt, synthetic construct
  source 1..23: mol_type = other RNA
  modified_base 1^2: mod_base = OTHER; note = Phosphorothioate Linkage
  modified_base 2^3: mod_base = OTHER; note = Phosphorothioate Linkage
  modified_base 21^22: mod_base = OTHER; note = Phosphorothioate Linkage
  modified_base 22^23: mod_base = OTHER; note = Phosphorothioate Linkage
  modified_base 2: mod_base = OTHER; note = 2&apos;-fluoronucleotide
  modified_base 4..5: mod_base = OTHER; note = 2&apos;-fluoronucleotide
  modified_base 8: mod_base = OTHER; note = 2&apos;-fluoronucleotide
  modified_base 10: mod_base = OTHER; note = 2&apos;-fluoronucleotide
  modified_base 12: mod_base = OTHER; note = 2&apos;-fluoronucleotide
  modified_base 14: mod_base = OTHER; note = 2&apos;-fluoronucleotide
  modified_base 16: mod_base = OTHER; note = 2&apos;-fluoronucleotide
  modified_base 18: mod_base = OTHER; note = 2&apos;-fluoronucleotide
  modified_base 1: mod_base = OTHER; note = 2&apos;-fluoronucleotide
  modified_base 3: mod_base = OTHER; note = 2&apos;-O-methylnucleotide
  modified_base 7: mod_base = OTHER; note = 2&apos;-O-methylnucleotide
  modified_base 9: mod_base = OTHER; note = 2&apos;-O-methylnucleotide
  modified_base 11: mod_base = OTHER; note = 2&apos;-O-methylnucleotide
  modified_base 13: mod_base = OTHER; note = 2&apos;-O-methylnucleotide
  modified_base 15: mod_base = OTHER; note = 2&apos;-O-methylnucleotide
  modified_base 17: mod_base = OTHER; note = 2&apos;-O-methylnucleotide
  modified_base 19..23: mod_base = OTHER; note = 2&apos;-O-methylnucleotide
  modified_base 6: mod_base = OTHER; note = 2&apos;-O-methylnucleotide
acaaaagcaaaacaggtctagaa

SEQ ID NO: 14 - RNA, 21 nt, synthetic construct
  source 1..21: mol_type = other RNA
  modified_base 1^2: mod_base = OTHER; note = Phosphorothioate Linkage
  modified_base 2^3: mod_base = OTHER; note = Phosphorothioate Linkage
  modified_base 7: mod_base = OTHER; note = 2&apos;-fluoronucleotide
  modified_base 9: mod_base = OTHER; note = 2&apos;-fluoronucleotide
  modified_base 1..6: mod_base = OTHER; note = 2&apos;-O-methylnucleotide
  modified_base 8: mod_base = OTHER; note = 2&apos;-O-methylnucleotide
  modified_base 12..21: mod_base = OTHER; note = 2&apos;-O-methylnucleotide
  modified_base 11: mod_base = OTHER; note = 2&apos;-deoxythymidine
  modified_base 10: mod_base = OTHER; note = 2&apos;-O-methylnucleotide
ctagacctgttttgcttttgt

SEQ ID NO: 15 - RNA, 21 nt, synthetic construct
  source 1..21: mol_type = other RNA
  modified_base 1^2: mod_base = OTHER; note = Phosphorothioate Linkage
  modified_base 1: mod_base = OTHER; note = 2&apos;-fluoronucleotide
  modified_base 3: mod_base = OTHER; note = 2&apos;-fluoronucleotide
  modified_base 7: mod_base = OTHER; note = 2&apos;-fluoronucleotide
  modified_base 9..11: mod_base = OTHER; note = 2&apos;-fluoronucleotide
  modified_base 2: mod_base = OTHER; note = 2&apos;-O-methylnucleotide
  modified_base 4..6: mod_base = OTHER; note = 2&apos;-O-methylnucleotide
  modified_base 8: mod_base = OTHER; note = 2&apos;-O-methylnucleotide
  modified_base 12..21: mod_base = OTHER; note = 2&apos;-O-methylnucleotide
ctagacctgttttgcttttgt

SEQ ID NO: 16 - RNA, 25 nt, synthetic construct
  source 1..25: mol_type = other RNA
  modified_base 1^2: mod_base = OTHER; note = Phosphorothioate Linkage
  modified_base 2^3: mod_base = OTHER; note = Phosphorothioate Linkage
  modified_base 21^22: mod_base = OTHER; note = Phosphorothioate Linkage
  modified_base 22^23: mod_base = OTHER; note = Phosphorothioate Linkage
  modified_base 2: mod_base = OTHER; note = 2&apos;-fluoronucleotide
  modified_base 4..6: mod_base = OTHER; note = 2&apos;-fluoronucleotide
  modified_base 8: mod_base = OTHER; note = 2&apos;-fluoronucleotide
  modified_base 10: mod_base = OTHER; note = 2&apos;-fluoronucleotide
  modified_base 12: mod_base = OTHER; note = 2&apos;-fluoronucleotide
  modified_base 14: mod_base = OTHER; note = 2&apos;-fluoronucleotide
  modified_base 16: mod_base = OTHER; note = 2&apos;-fluoronucleotide
  modified_base 18: mod_base = OTHER; note = 2&apos;-fluoronucleotide
  modified_base 1: mod_base = OTHER; note = 2&apos;-fluoronucleotide
  modified_base 3: mod_base = OTHER; note = 2&apos;-O-methylnucleotide
  modified_base 7: mod_base = OTHER; note = 2&apos;-O-methylnucleotide
  modified_base 9: mod_base = OTHER; note = 2&apos;-O-methylnucleotide
  modified_base 11: mod_base = OTHER; note = 2&apos;-O-methylnucleotide
  modified_base 13: mod_base = OTHER; note = 2&apos;-O-methylnucleotide
  modified_base 15: mod_base = OTHER; note = 2&apos;-O-methylnucleotide
  modified_base 17: mod_base = OTHER; note = 2&apos;-O-methylnucleotide
  modified_base 19..23: mod_base = OTHER; note = 2&apos;-O-methylnucleotide
  modified_base 24..25: mod_base = OTHER; note = 2&apos;-deoxythymidine
acaaaagcaaaacaggtctagaatt

SEQ ID NO: 17 - RNA, 25 nt, synthetic construct
  source 1..25: mol_type = other RNA
  modified_base 1^2: mod_base = OTHER; note = Phosphorothioate Linkage
  modified_base 2^3: mod_base = OTHER; note = Phosphorothioate Linkage
  modified_base 21^22: mod_base = OTHER; note = Phosphorothioate Linkage
  modified_base 22^23: mod_base = OTHER; note = Phosphorothioate Linkage
  modified_base 2: mod_base = OTHER; note = 2&apos;-fluoronucleotide
  modified_base 4..6: mod_base = OTHER; note = 2&apos;-fluoronucleotide
  modified_base 8: mod_base = OTHER; note = 2&apos;-fluoronucleotide
  modified_base 10: mod_base = OTHER; note = 2&apos;-fluoronucleotide
  modified_base 12: mod_base = OTHER; note = 2&apos;-fluoronucleotide
  modified_base 14: mod_base = OTHER; note = 2&apos;-fluoronucleotide
  modified_base 16: mod_base = OTHER; note = 2&apos;-fluoronucleotide
  modified_base 18: mod_base = OTHER; note = 2&apos;-fluoronucleotide
  modified_base 1: mod_base = OTHER; note = 2&apos;-fluoronucleotide
  modified_base 3: mod_base = OTHER; note = 2&apos;-O-methylnucleotide
  modified_base 7: mod_base = OTHER; note = 2&apos;-O-methylnucleotide
  modified_base 9: mod_base = OTHER; note = 2&apos;-O-methylnucleotide
  modified_base 11: mod_base = OTHER; note = 2&apos;-O-methylnucleotide
  modified_base 13: mod_base = OTHER; note = 2&apos;-O-methylnucleotide
  modified_base 15: mod_base = OTHER; note = 2&apos;-O-methylnucleotide
  modified_base 17: mod_base = OTHER; note = 2&apos;-O-methylnucleotide
  modified_base 19..23: mod_base = OTHER; note = 2&apos;-O-methylnucleotide
  modified_base 24..25: mod_base = OTHER; note = 2&apos;-O-methylnucleotide
acaaaagcaaaacaggtctagaatt

SEQ ID NO: 18 - RNA, 21 nt, synthetic construct
  source 1..21: mol_type = other RNA
  modified_base 1^2: mod_base = OTHER; note = Phosphorothioate Linkage
  modified_base 2^3: mod_base = OTHER; note = Phosphorothioate Linkage
  modified_base 1: mod_base = OTHER; note = 2&apos;-fluoronucleotide
  modified_base 3: mod_base = OTHER; note = 2&apos;-fluoronucleotide
  modified_base 7: mod_base = OTHER; note = 2&apos;-fluoronucleotide
  modified_base 9..11: mod_base = OTHER; note = 2&apos;-fluoronucleotide
  modified_base 2: mod_base = OTHER; note = 2&apos;-O-methylnucleotide
  modified_base 4..6: mod_base = OTHER; note = 2&apos;-O-methylnucleotide
  modified_base 8: mod_base = OTHER; note = 2&apos;-O-methylnucleotide
  modified_base 12..21: mod_base = OTHER; note = 2&apos;-O-methylnucleotide
  modified_base 1: mod_base = OTHER; note = Conjugated with a reversed abasic linker
ctagacctgttttgcttttgt

SEQ ID NO: 19 - RNA, 21 nt, synthetic construct
  source 1..21: mol_type = other RNA
  modified_base 1^2: mod_base = OTHER; note = Phosphorothioate Linkage
  modified_base 2^3: mod_base = OTHER; note = Phosphorothioate Linkage
  modified_base 1: mod_base = OTHER; note = 2&apos;-fluoronucleotide
  modified_base 3: mod_base = OTHER; note = 2&apos;-fluoronucleotide
  modified_base 7: mod_base = OTHER; note = 2&apos;-fluoronucleotide
  modified_base 9..11: mod_base = OTHER; note = 2&apos;-fluoronucleotide
  modified_base 2: mod_base = OTHER; note = 2&apos;-O-methylnucleotide
  modified_base 4..6: mod_base = OTHER; note = 2&apos;-O-methylnucleotide
  modified_base 8: mod_base = OTHER; note = 2&apos;-O-methylnucleotide
  modified_base 12..21: mod_base = OTHER; note = 2&apos;-O-methylnucleotide
  modified_base 1: mod_base = OTHER; note = Conjugated with a reversed abasic linker
  modified_base 21: mod_base = OTHER; note = Conjugated with a reversed abasic linker
ctagacctgttttgcttttgt

SEQ ID NO: 20 - RNA, 21 nt, synthetic construct
  source 1..21: mol_type = other RNA
  modified_base 1^2: mod_base = OTHER; note = Phosphorothioate Linkage
  modified_base 2^3: mod_base = OTHER; note = Phosphorothioate Linkage
  modified_base 1: mod_base = OTHER; note = 2&apos;-fluoronucleotide
  modified_base 3: mod_base = OTHER; note = 2&apos;-fluoronucleotide
  modified_base 7: mod_base = OTHER; note = 2&apos;-fluoronucleotide
  modified_base 9..11: mod_base = OTHER; note = 2&apos;-fluoronucleotide
  modified_base 2: mod_base = OTHER; note = 2&apos;-O-methylnucleotide
  modified_base 4..6: mod_base = OTHER; note = 2&apos;-O-methylnucleotide
  modified_base 8: mod_base = OTHER; note = 2&apos;-O-methylnucleotide
  modified_base 12..21: mod_base = OTHER; note = 2&apos;-O-methylnucleotide
  modified_base 21: mod_base = OTHER; note = Conjugated with a reversed abasic linker
ctagacctgttttgcttttgt

SEQ ID NO: 21 - RNA, 21 nt, synthetic construct
  source 1..21: mol_type = other RNA
  modified_base 1^2: mod_base = OTHER; note = Phosphorothioate Linkage
  modified_base 1: mod_base = OTHER; note = 2&apos;-fluoronucleotide
  modified_base 3: mod_base = OTHER; note = 2&apos;-fluoronucleotide
  modified_base 7: mod_base = OTHER; note = 2&apos;-fluoronucleotide
  modified_base 9..11: mod_base = OTHER; note = 2&apos;-fluoronucleotide
  modified_base 2: mod_base = OTHER; note = 2&apos;-O-methylnucleotide
  modified_base 4..6: mod_base = OTHER; note = 2&apos;-O-methylnucleotide
  modified_base 8: mod_base = OTHER; note = 2&apos;-O-methylnucleotide
  modified_base 15..21: mod_base = OTHER; note = 2&apos;-O-methylnucleotide
  modified_base 12..13: mod_base = OTHER; note = 2&apos;-O-methylnucleotide
  modified_base 14: mod_base = OTHER; note = 2&apos;-fluoronucleotide
ctagacctgttttgcttttgt

SEQ ID NO: 22 - RNA, 21 nt, synthetic construct
  source 1..21: mol_type = other RNA
  modified_base 1^2: mod_base = OTHER; note = Phosphorothioate Linkage
  modified_base 2^3: mod_base = OTHER; note = Phosphorothioate Linkage
  modified_base 1: mod_base = OTHER; note = 2&apos;-fluoronucleotide
  modified_base 3: mod_base = OTHER; note = 2&apos;-fluoronucleotide
  modified_base 7: mod_base = OTHER; note = 2&apos;-fluoronucleotide
  modified_base 9..11: mod_base = OTHER; note = 2&apos;-fluoronucleotide
  modified_base 2: mod_base = OTHER; note = 2&apos;-O-methylnucleotide
  modified_base 4..6: mod_base = OTHER; note = 2&apos;-O-methylnucleotide
  modified_base 8: mod_base = OTHER; note = 2&apos;-O-methylnucleotide
  modified_base 15..21: mod_base = OTHER; note = 2&apos;-O-methylnucleotide
  modified_base 12..13: mod_base = OTHER; note = 2&apos;-O-methylnucleotide
  modified_base 14: mod_base = OTHER; note = 2&apos;-fluoronucleotide
  modified_base 1: mod_base = OTHER; note = Conjugated with a reversed abasic linker
ctagacctgttttgcttttgt

SEQ ID NO: 23 - RNA, 21 nt, synthetic construct
  source 1..21: mol_type = other RNA
  modified_base 1^2: mod_base = OTHER; note = Phosphorothioate Linkage
  modified_base 2^3: mod_base = OTHER; note = Phosphorothioate Linkage
  modified_base 1: mod_base = OTHER; note = 2&apos;-fluoronucleotide
  modified_base 3: mod_base = OTHER; note = 2&apos;-fluoronucleotide
  modified_base 7: mod_base = OTHER; note = 2&apos;-fluoronucleotide
  modified_base 9..11: mod_base = OTHER; note = 2&apos;-fluoronucleotide
  modified_base 2: mod_base = OTHER; note = 2&apos;-O-methylnucleotide
  modified_base 4..6: mod_base = OTHER; note = 2&apos;-O-methylnucleotide
  modified_base 8: mod_base = OTHER; note = 2&apos;-O-methylnucleotide
  modified_base 15..21: mod_base = OTHER; note = 2&apos;-O-methylnucleotide
  modified_base 12..13: mod_base = OTHER; note = 2&apos;-O-methylnucleotide
  modified_base 14: mod_base = OTHER; note = 2&apos;-fluoronucleotide
  modified_base 1: mod_base = OTHER; note = Conjugated with a reversed abasic linker
  modified_base 21: mod_base = OTHER; note = Conjugated with a reversed abasic linker
ctagacctgttttgcttttgt

SEQ ID NO: 24 - RNA, 21 nt, synthetic construct
  source 1..21: mol_type = other RNA
  modified_base 1^2: mod_base = OTHER; note = Phosphorothioate Linkage
  modified_base 2^3: mod_base = OTHER; note = Phosphorothioate Linkage
  modified_base 1: mod_base = OTHER; note = 2&apos;-fluoronucleotide
  modified_base 3: mod_base = OTHER; note = 2&apos;-fluoronucleotide
  modified_base 7: mod_base = OTHER; note = 2&apos;-fluoronucleotide
  modified_base 9..11: mod_base = OTHER; note = 2&apos;-fluoronucleotide
  modified_base 2: mod_base = OTHER; note = 2&apos;-O-methylnucleotide
  modified_base 4..6: mod_base = OTHER; note = 2&apos;-O-methylnucleotide
  modified_base 8: mod_base = OTHER; note = 2&apos;-O-methylnucleotide
  modified_base 15..21: mod_base = OTHER; note = 2&apos;-O-methylnucleotide
  modified_base 12..13: mod_base = OTHER; note = 2&apos;-O-methylnucleotide
  modified_base 14: mod_base = OTHER; note = 2&apos;-fluoronucleotide
  modified_base 21: mod_base = OTHER; note = Conjugated with a reversed abasic linker
ctagacctgttttgcttttgt

SEQ ID NO: 25 - RNA, 21 nt, synthetic construct
  source 1..21: mol_type = other RNA
  modified_base 1^2: mod_base = OTHER; note = Phosphorothioate Linkage
  modified_base 1: mod_base = OTHER; note = 2&apos;-fluoronucleotide
  modified_base 3: mod_base = OTHER; note = 2&apos;-fluoronucleotide
  modified_base 7: mod_base = OTHER; note = 2&apos;-fluoronucleotide
  modified_base 9..11: mod_base = OTHER; note = 2&apos;-fluoronucleotide
  modified_base 2: mod_base = OTHER; note = 2&apos;-O-methylnucleotide
  modified_base 4..6: mod_base = OTHER; note = 2&apos;-O-methylnucleotide
  modified_base 8: mod_base = OTHER; note = 2&apos;-fluoronucleotide
  modified_base 15..21: mod_base = OTHER; note = 2&apos;-O-methylnucleotide
  modified_base 12..13: mod_base = OTHER; note = 2&apos;-O-methylnucleotide
  modified_base 14: mod_base = OTHER; note = 2&apos;-fluoronucleotide
ctagacctgttttgcttttgt

SEQ ID NO: 26 - RNA, 21 nt, synthetic construct
  source 1..21: mol_type = other RNA
  modified_base 1^2: mod_base = OTHER; note = Phosphorothioate Linkage
  modified_base 2^3: mod_base = OTHER; note = Phosphorothioate Linkage
  modified_base 1: mod_base = OTHER; note = 2&apos;-fluoronucleotide
  modified_base 3: mod_base = OTHER; note = 2&apos;-fluoronucleotide
  modified_base 7: mod_base = OTHER; note = 2&apos;-fluoronucleotide
  modified_base 9..11: mod_base = OTHER; note = 2&apos;-fluoronucleotide
  modified_base 2: mod_base = OTHER; note = 2&apos;-O-methylnucleotide
  modified_base 4..6: mod_base = OTHER; note = 2&apos;-O-methylnucleotide
  modified_base 8: mod_base = OTHER; note = 2&apos;-fluoronucleotide
  modified_base 15..21: mod_base = OTHER; note = 2&apos;-O-methylnucleotide
  modified_base 12..13: mod_base = OTHER; note = 2&apos;-O-methylnucleotide
  modified_base 14: mod_base = OTHER; note = 2&apos;-fluoronucleotide
  modified_base 1: mod_base = OTHER; note = Conjugated with a reversed abasic linker
ctagacctgttttgcttttgt

SEQ ID NO: 27 - RNA, 21 nt, synthetic construct
  source 1..21: mol_type = other RNA
  modified_base 1^2: mod_base = OTHER; note = Phosphorothioate Linkage
  modified_base 2^3: mod_base = OTHER; note = Phosphorothioate Linkage
  modified_base 1: mod_base = OTHER; note = 2&apos;-fluoronucleotide
  modified_base 3: mod_base = OTHER; note = 2&apos;-fluoronucleotide
  modified_base 7: mod_base = OTHER; note = 2&apos;-fluoronucleotide
  modified_base 9..11: mod_base = OTHER; note = 2&apos;-fluoronucleotide
  modified_base 2: mod_base = OTHER; note = 2&apos;-O-methylnucleotide
  modified_base 4..6: mod_base = OTHER; note = 2&apos;-O-methylnucleotide
  modified_base 8: mod_base = OTHER; note = 2&apos;-fluoronucleotide
  modified_base 15..21: mod_base = OTHER; note = 2&apos;-O-methylnucleotide
  modified_base 12..13: mod_base = OTHER; note = 2&apos;-O-methylnucleotide
  modified_base 14: mod_base = OTHER; note = 2&apos;-fluoronucleotide
  modified_base 1: mod_base = OTHER; note = Conjugated with a reversed abasic linker
  modified_base 21: mod_base = OTHER; note = Conjugated with a reversed abasic linker
ctagacctgttttgcttttgt

SEQ ID NO: 28 - RNA, 21 nt, synthetic construct
  source 1..21: mol_type = other RNA
  modified_base 1^2: mod_base = OTHER; note = Phosphorothioate Linkage
  modified_base 2^3: mod_base = OTHER; note = Phosphorothioate Linkage
  modified_base 1: mod_base = OTHER; note = 2&apos;-fluoronucleotide
  modified_base 3: mod_base = OTHER; note = 2&apos;-fluoronucleotide
  modified_base 7: mod_base = OTHER; note = 2&apos;-fluoronucleotide
  modified_base 9..11: mod_base = OTHER; note = 2&apos;-fluoronucleotide
  modified_base 2: mod_base = OTHER; note = 2&apos;-O-methylnucleotide
  modified_base 4..6: mod_base = OTHER; note = 2&apos;-O-methylnucleotide
  modified_base 8: mod_base = OTHER; note = 2&apos;-fluoronucleotide
  modified_base 15..21: mod_base = OTHER; note = 2&apos;-O-methylnucleotide
  modified_base 12..13: mod_base = OTHER; note = 2&apos;-O-methylnucleotide
  modified_base 14: mod_base = OTHER; note = 2&apos;-fluoronucleotide
  modified_base 21: mod_base = OTHER; note = Conjugated with a reversed abasic linker
ctagacctgttttgcttttgt

SEQ ID NO: 29 - RNA, 21 nt, synthetic construct
  source 1..21: mol_type = other RNA
  modified_base 1^2: mod_base = OTHER; note = Phosphorothioate Linkage
  modified_base 1: mod_base = OTHER; note = 2&apos;-fluoronucleotide
  modified_base 3: mod_base = OTHER; note = 2&apos;-fluoronucleotide
  modified_base 7: mod_base = OTHER; note = 2&apos;-fluoronucleotide
  modified_base 9: mod_base = OTHER; note = 2&apos;-fluoronucleotide
  modified_base 2: mod_base = OTHER; note = 2&apos;-O-methylnucleotide
  modified_base 4..6: mod_base = OTHER; note = 2&apos;-O-methylnucleotide
  modified_base 8: mod_base = OTHER; note = 2&apos;-O-methylnucleotide
  modified_base 12..21: mod_base = OTHER; note = 2&apos;-O-methylnucleotide
  modified_base 11: mod_base = OTHER; note = 2&apos;-deoxythymidine
  modified_base 10: mod_base = OTHER; note = 2&apos;-O-methylnucleotide
ctagacctgttttgcttttgt

SEQ ID NO: 30 - RNA, 21 nt, synthetic construct
  source 1..21: mol_type = other RNA
  modified_base 1^2: mod_base = OTHER; note = Phosphorothioate Linkage
  modified_base 2^3: mod_base = OTHER; note = Phosphorothioate Linkage
  modified_base 1: mod_base = OTHER; note = 2&apos;-fluoronucleotide
  modified_base 3: mod_base = OTHER; note = 2&apos;-fluoronucleotide
  modified_base 7: mod_base = OTHER; note = 2&apos;-fluoronucleotide
  modified_base 9: mod_base = OTHER; note = 2&apos;-fluoronucleotide
  modified_base 2: mod_base = OTHER; note = 2&apos;-O-methylnucleotide
  modified_base 4..6: mod_base = OTHER; note = 2&apos;-O-methylnucleotide
  modified_base 8: mod_base = OTHER; note = 2&apos;-O-methylnucleotide
  modified_base 12..21: mod_base = OTHER; note = 2&apos;-O-methylnucleotide
  modified_base 11: mod_base = OTHER; note = 2&apos;-deoxythymidine
  modified_base 10: mod_base = OTHER; note = 2&apos;-O-methylnucleotide
  modified_base 1: mod_base = OTHER; note = Conjugated with a reversed abasic linker
ctagacctgttttgcttttgt

SEQ ID NO: 31 - RNA, 21 nt, synthetic construct
  source 1..21: mol_type = other RNA
  modified_base 1^2: mod_base = OTHER; note = Phosphorothioate Linkage
  modified_base 2^3: mod_base = OTHER; note = Phosphorothioate Linkage
  modified_base 1: mod_base = OTHER; note = 2&apos;-fluoronucleotide
  modified_base 3: mod_base = OTHER; note = 2&apos;-fluoronucleotide
  modified_base 7: mod_base = OTHER; note = 2&apos;-fluoronucleotide
  modified_base 9: mod_base = OTHER; note = 2&apos;-fluoronucleotide
  modified_base 2: mod_base = OTHER; note = 2&apos;-O-methylnucleotide
  modified_base 4..6: mod_base = OTHER; note = 2&apos;-O-methylnucleotide
  modified_base 8: mod_base = OTHER; note = 2&apos;-O-methylnucleotide
  modified_base 12..21: mod_base = OTHER; note = 2&apos;-O-methylnucleotide
  modified_base 11: mod_base = OTHER; note = 2&apos;-deoxythymidine
  modified_base 10: mod_base = OTHER; note = 2&apos;-O-methylnucleotide
  modified_base 1: mod_base = OTHER; note = Conjugated with a reversed abasic linker
  modified_base 21: mod_base = OTHER; note = Conjugated with a reversed abasic linker
ctagacctgttttgcttttgt

SEQ ID NO: 32 - RNA, 21 nt, synthetic construct
  source 1..21: mol_type = other RNA
  modified_base 1^2: mod_base = OTHER; note = Phosphorothioate Linkage
  modified_base 2^3: mod_base = OTHER; note = Phosphorothioate Linkage
  modified_base 1: mod_base = OTHER; note = 2&apos;-fluoronucleotide
  modified_base 3: mod_base = OTHER; note = 2&apos;-fluoronucleotide
  modified_base 7: mod_base = OTHER; note = 2&apos;-fluoronucleotide
  modified_base 9: mod_base = OTHER; note = 2&apos;-fluoronucleotide
  modified_base 2: mod_base = OTHER; note = 2&apos;-O-methylnucleotide
  modified_base 4..6: mod_base = OTHER; note = 2&apos;-O-methylnucleotide
  modified_base 8: mod_base = OTHER; note = 2&apos;-O-methylnucleotide
  modified_base 12..21: mod_base = OTHER; note = 2&apos;-O-methylnucleotide
  modified_base 11: mod_base = OTHER; note = 2&apos;-deoxythymidine
  modified_base 10: mod_base = OTHER; note = 2&apos;-O-methylnucleotide
  modified_base 21: mod_base = OTHER; note = Conjugated with a reversed abasic linker
ctagacctgttttgcttttgt

SEQ ID NO: 33 - RNA, 21 nt, synthetic construct
  source 1..21: mol_type = other RNA
  modified_base 1^2: mod_base = OTHER; note = Phosphorothioate Linkage
  modified_base 1: mod_base = OTHER; note = 2&apos;-fluoronucleotide
  modified_base 3: mod_base = OTHER; note = 2&apos;-fluoronucleotide
  modified_base 7: mod_base = OTHER; note = 2&apos;-fluoronucleotide
  modified_base 9: mod_base = OTHER; note = 2&apos;-fluoronucleotide
  modified_base 2: mod_base = OTHER; note = 2&apos;-O-methylnucleotide
  modified_base 4..6: mod_base = OTHER; note = 2&apos;-O-methylnucleotide
  modified_base 8: mod_base = OTHER; note = 2&apos;-O-methylnucleotide
  modified_base 12..13: mod_base = OTHER; note = 2&apos;-O-methylnucleotide
  modified_base 11: mod_base = OTHER; note = 2&apos;-deoxythymidine
  modified_base 10: mod_base = OTHER; note = 2&apos;-O-methylnucleotide
  modified_base 14: mod_base = OTHER; note = 2&apos;-fluoronucleotide
  modified_base 15..21: mod_base = OTHER; note = 2&apos;-O-methylnucleotide
ctagacctgttttgcttttgt

SEQ ID NO: 34 - RNA, 21 nt, synthetic construct
  source 1..21: mol_type = other RNA
  modified_base 1^2: mod_base = OTHER; note = Phosphorothioate Linkage
  modified_base 2^3: mod_base = OTHER; note = Phosphorothioate Linkage
  modified_base 1: mod_base = OTHER; note = 2&apos;-fluoronucleotide
  modified_base 3: mod_base = OTHER; note = 2&apos;-fluoronucleotide
  modified_base 7: mod_base = OTHER; note = 2&apos;-fluoronucleotide
  modified_base 9: mod_base = OTHER; note = 2&apos;-fluoronucleotide
  modified_base 2: mod_base = OTHER; note = 2&apos;-O-methylnucleotide
  modified_base 4..6: mod_base = OTHER; note = 2&apos;-O-methylnucleotide
  modified_base 8: mod_base = OTHER; note = 2&apos;-O-methylnucleotide
  modified_base 12..13: mod_base = OTHER; note = 2&apos;-O-methylnucleotide
  modified_base 11: mod_base = OTHER; note = 2&apos;-deoxythymidine
  modified_base 10: mod_base = OTHER; note = 2&apos;-O-methylnucleotide
  modified_base 14: mod_base = OTHER; note = 2&apos;-fluoronucleotide
  modified_base 15..21: mod_base = OTHER; note = 2&apos;-O-methylnucleotide
  modified_base 1: mod_base = OTHER; note = Conjugated with a reversed abasic linker
ctagacctgttttgcttttgt

SEQ ID NO: 35 - RNA, 21 nt, synthetic construct
  source 1..21: mol_type = other RNA
  modified_base 1^2: mod_base = OTHER; note = Phosphorothioate Linkage
  modified_base 2^3: mod_base = OTHER; note = Phosphorothioate Linkage
  modified_base 1: mod_base = OTHER; note = 2&apos;-fluoronucleotide
  modified_base 3: mod_base = OTHER; note = 2&apos;-fluoronucleotide
  modified_base 7: mod_base = OTHER; note = 2&apos;-fluoronucleotide
  modified_base 9: mod_base = OTHER; note = 2&apos;-fluoronucleotide
  modified_base 2: mod_base = OTHER; note = 2&apos;-O-methylnucleotide
  modified_base 4..6: mod_base = OTHER; note = 2&apos;-O-methylnucleotide
  modified_base 8: mod_base = OTHER; note = 2&apos;-O-methylnucleotide
  modified_base 12..13: mod_base = OTHER; note = 2&apos;-O-methylnucleotide
  modified_base 11: mod_base = OTHER; note = 2&apos;-deoxythymidine
  modified_base 10: mod_base = OTHER; note = 2&apos;-O-methylnucleotide
  modified_base 14: mod_base = OTHER; note = 2&apos;-fluoronucleotide
  modified_base 15..21: mod_base = OTHER; note = 2&apos;-O-methylnucleotide
  modified_base 1: mod_base = OTHER; note = Conjugated with a reversed abasic linker
  modified_base 21: mod_base = OTHER; note = Conjugated with a reversed abasic linker
ctagacctgttttgcttttgt

SEQ ID NO: 36 - RNA, 21 nt, synthetic construct
  source 1..21: mol_type = other RNA
  modified_base 1^2: mod_base = OTHER; note = Phosphorothioate Linkage
  modified_base 2^3: mod_base = OTHER; note = Phosphorothioate Linkage
  modified_base 1: mod_base = OTHER; note = 2&apos;-fluoronucleotide
  modified_base 3: mod_base = OTHER; note = 2&apos;-fluoronucleotide
  modified_base 7: mod_base = OTHER; note = 2&apos;-fluoronucleotide
  modified_base 9: mod_base = OTHER; note = 2&apos;-fluoronucleotide
  modified_base 2: mod_base = OTHER; note = 2&apos;-O-methylnucleotide
  modified_base 4..6: mod_base = OTHER; note = 2&apos;-O-methylnucleotide
  modified_base 8: mod_base = OTHER; note = 2&apos;-O-methylnucleotide
  modified_base 12..13: mod_base = OTHER; note = 2&apos;-O-methylnucleotide
  modified_base 11: mod_base = OTHER; note = 2&apos;-deoxythymidine
  modified_base 10: mod_base = OTHER; note = 2&apos;-O-methylnucleotide
  modified_base 14: mod_base = OTHER; note = 2&apos;-fluoronucleotide
  modified_base 15..21: mod_base = OTHER; note = 2&apos;-O-methylnucleotide
  modified_base 21: mod_base = OTHER; note = Conjugated with a reversed abasic linker
ctagacctgttttgcttttgt

SEQ ID NO: 37 - RNA, 21 nt, synthetic construct
  source 1..21: mol_type = other RNA
  modified_base 1^2: mod_base = OTHER; note = Phosphorothioate Linkage
  modified_base 1: mod_base = OTHER; note = 2&apos;-fluoronucleotide
  modified_base 3: mod_base = OTHER; note = 2&apos;-fluoronucleotide
  modified_base 7: mod_base = OTHER; note = 2&apos;-fluoronucleotide
  modified_base 9: mod_base = OTHER; note = 2&apos;-fluoronucleotide
  modified_base 2: mod_base = OTHER; note = 2&apos;-O-methylnucleotide
  modified_base 4..6: mod_base = OTHER; note = 2&apos;-O-methylnucleotide
  modified_base 8: mod_base = OTHER; note = 2&apos;-fluoronucleotide
  modified_base 12..13: mod_base = OTHER; note = 2&apos;-O-methylnucleotide
  modified_base 11: mod_base = OTHER; note = 2&apos;-deoxythymidine
  modified_base 10: mod_base = OTHER; note = 2&apos;-O-methylnucleotide
  modified_base 14: mod_base = OTHER; note = 2&apos;-fluoronucleotide
  modified_base 15..21: mod_base = OTHER; note = 2&apos;-O-methylnucleotide
ctagacctgttttgcttttgt

SEQ ID NO: 38 - RNA, 21 nt, synthetic construct
  source 1..21: mol_type = other RNA
  modified_base 1^2: mod_base = OTHER; note = Phosphorothioate Linkage
  modified_base 2^3: mod_base = OTHER; note = Phosphorothioate Linkage
  modified_base 1: mod_base = OTHER; note = 2&apos;-fluoronucleotide
  modified_base 3: mod_base = OTHER; note = 2&apos;-fluoronucleotide
  modified_base 7: mod_base = OTHER; note = 2&apos;-fluoronucleotide
  modified_base 9: mod_base = OTHER; note = 2&apos;-fluoronucleotide
  modified_base 2: mod_base = OTHER; note = 2&apos;-O-methylnucleotide
  modified_base 4..6: mod_base = OTHER; note = 2&apos;-O-methylnucleotide
  modified_base 8: mod_base = OTHER; note = 2&apos;-fluoronucleotide
  modified_base 12..13: mod_base = OTHER; note = 2&apos;-O-methylnucleotide
  modified_base 11: mod_base = OTHER; note = 2&apos;-deoxythymidine
  modified_base 10: mod_base = OTHER; note = 2&apos;-O-methylnucleotide
  modified_base 14: mod_base = OTHER; note = 2&apos;-fluoronucleotide
  modified_base 15..21: mod_base = OTHER; note = 2&apos;-O-methylnucleotide
  modified_base 1: mod_base = OTHER; note = Conjugated with a reversed abasic linker
ctagacctgttttgcttttgt

SEQ ID NO: 39 - RNA, 21 nt, synthetic construct
  source 1..21: mol_type = other RNA
  modified_base 1^2: mod_base = OTHER; note = Phosphorothioate Linkage
  modified_base 2^3: mod_base = OTHER; note = Phosphorothioate Linkage
  modified_base 1: mod_base = OTHER; note = 2&apos;-fluoronucleotide
  modified_base 3: mod_base = OTHER; note = 2&apos;-fluoronucleotide
  modified_base 7: mod_base = OTHER; note = 2&apos;-fluoronucleotide
  modified_base 9: mod_base = OTHER; note = 2&apos;-fluoronucleotide
  modified_base 2: mod_base = OTHER; note = 2&apos;-O-methylnucleotide
  modified_base 4..6: mod_base = OTHER; note = 2&apos;-O-methylnucleotide
  modified_base 8: mod_base = OTHER; note = 2&apos;-fluoronucleotide
  modified_base 12..13: mod_base = OTHER; note = 2&apos;-O-methylnucleotide
  modified_base 11: mod_base = OTHER; note = 2&apos;-deoxythymidine
  modified_base 10: mod_base = OTHER; note = 2&apos;-O-methylnucleotide
  modified_base 14: mod_base = OTHER; note = 2&apos;-fluoronucleotide
  modified_base 15..21: mod_base = OTHER; note = 2&apos;-O-methylnucleotide
  modified_base 1: mod_base = OTHER; note = Conjugated with a reversed abasic linker
  modified_base 21: mod_base = OTHER; note = Conjugated with a reversed abasic linker
ctagacctgttttgcttttgt

SEQ ID NO: 40 - RNA, 21 nt, synthetic construct
  source 1..21: mol_type = other RNA
  modified_base 1^2: mod_base = OTHER; note = Phosphorothioate Linkage
  modified_base 2^3: mod_base = OTHER; note = Phosphorothioate Linkage
  modified_base 1: mod_base = OTHER; note = 2&apos;-fluoronucleotide
  modified_base 3: mod_base = OTHER; note = 2&apos;-fluoronucleotide
  modified_base 7: mod_base = OTHER; note = 2&apos;-fluoronucleotide
  modified_base 9: mod_base = OTHER; note = 2&apos;-fluoronucleotide
  modified_base 2: mod_base = OTHER; note = 2&apos;-O-methylnucleotide
  modified_base 4..6: mod_base = OTHER; note = 2&apos;-O-methylnucleotide
  modified_base 8: mod_base = OTHER; note = 2&apos;-fluoronucleotide
  modified_base 12..13: mod_base = OTHER; note = 2&apos;-O-methylnucleotide
  modified_base 11: mod_base = OTHER; note = 2&apos;-deoxythymidine
  modified_base 10: mod_base = OTHER; note = 2&apos;-O-methylnucleotide
  modified_base 14: mod_base = OTHER; note = 2&apos;-fluoronucleotide
  modified_base 15..21: mod_base = OTHER; note = 2&apos;-O-methylnucleotide
  modified_base 21: mod_base = OTHER; note = Conjugated with a reversed abasic linker
ctagacctgttttgcttttgt

SEQ ID NO: 41 - RNA, 21 nt, synthetic construct
  source 1..21: mol_type = other RNA
  modified_base 1^2: mod_base = OTHER; note = Phosphorothioate Linkage
  modified_base 1: mod_base = OTHER; note = 2&apos;-fluoronucleotide
  modified_base 3: mod_base = OTHER; note = 2&apos;-fluoronucleotide
  modified_base 7: mod_base = OTHER; note = 2&apos;-fluoronucleotide
  modified_base 9: mod_base = OTHER; note = 2&apos;-fluoronucleotide
  modified_base 2: mod_base = OTHER; note = 2&apos;-O-methylnucleotide
  modified_base 4..6: mod_base = OTHER; note = 2&apos;-O-methylnucleotide
  modified_base 8: mod_base = OTHER; note = 2&apos;-O-methylnucleotide
  modified_base 12..13: mod_base = OTHER; note = 2&apos;-O-methylnucleotide
  modified_base 11: mod_base = OTHER; note = 2&apos;-deoxythymidine
  modified_base 10: mod_base = OTHER; note = 2&apos;-fluoronucleotide
  modified_base 14: mod_base = OTHER; note = 2&apos;-fluoronucleotide
  modified_base 15..21: mod_base = OTHER; note = 2&apos;-O-methylnucleotide
ctagacctgttttgcttttgt

SEQ ID NO: 42 - RNA, 21 nt, synthetic construct
  source 1..21: mol_type = other RNA
  modified_base 1^2: mod_base = OTHER; note = Phosphorothioate Linkage
  modified_base 2^3: mod_base = OTHER; note = Phosphorothioate Linkage
  modified_base 1: mod_base = OTHER; note = 2&apos;-fluoronucleotide
  modified_base 3: mod_base = OTHER; note = 2&apos;-fluoronucleotide
  modified_base 7: mod_base = OTHER; note = 2&apos;-fluoronucleotide
  modified_base 9: mod_base = OTHER; note = 2&apos;-fluoronucleotide
  modified_base 2: mod_base = OTHER; note = 2&apos;-O-methylnucleotide
  modified_base 4..6: mod_base = OTHER; note = 2&apos;-O-methylnucleotide
  modified_base 8: mod_base = OTHER; note = 2&apos;-O-methylnucleotide
  modified_base 12..13: mod_base = OTHER; note = 2&apos;-O-methylnucleotide
  modified_base 11: mod_base = OTHER; note = 2&apos;-deoxythymidine
  modified_base 10: mod_base = OTHER; note = 2&apos;-fluoronucleotide
  modified_base 14: mod_base = OTHER; note = 2&apos;-fluoronucleotide
  modified_base 15..21: mod_base = OTHER; note = 2&apos;-O-methylnucleotide
  modified_base 1: mod_base = OTHER; note = Conjugated with a reversed abasic linker
ctagacctgttttgcttttgt

SEQ ID NO: 43 - RNA, 21 nt, synthetic construct
  source 1..21: mol_type = other RNA
  modified_base 1^2: mod_base = OTHER; note = Phosphorothioate Linkage
  modified_base 2^3: mod_base = OTHER; note = Phosphorothioate Linkage
  modified_base 1: mod_base = OTHER; note = 2&apos;-fluoronucleotide
  modified_base 3: mod_base = OTHER; note = 2&apos;-fluoronucleotide
  modified_base 7: mod_base = OTHER; note = 2&apos;-fluoronucleotide
  modified_base 9: mod_base = OTHER; note = 2&apos;-fluoronucleotide
  modified_base 2: mod_base = OTHER; note = 2&apos;-O-methylnucleotide
  modified_base 4..6: mod_base = OTHER; note = 2&apos;-O-methylnucleotide
  modified_base 8: mod_base = OTHER; note = 2&apos;-O-methylnucleotide
  modified_base 12..13: mod_base = OTHER; note = 2&apos;-O-methylnucleotide
  modified_base 11: mod_base = OTHER; note = 2&apos;-deoxythymidine
  modified_base 10: mod_base = OTHER; note = 2&apos;-fluoronucleotide
  modified_base 14: mod_base = OTHER; note = 2&apos;-fluoronucleotide
  modified_base 15..21: mod_base = OTHER; note = 2&apos;-O-methylnucleotide
  modified_base 1: mod_base = OTHER; note = Conjugated with a reversed abasic linker
  modified_base 21: mod_base = OTHER; note = Conjugated with a reversed abasic linker
ctagacctgttttgcttttgt

SEQ ID NO: 44 - RNA, 21 nt, synthetic construct
  source 1..21: mol_type = other RNA
  modified_base 1^2: mod_base = OTHER; note = Phosphorothioate Linkage
  modified_base 2^3: mod_base = OTHER; note = Phosphorothioate Linkage
  modified_base 1: mod_base = OTHER; note = 2&apos;-fluoronucleotide
  modified_base 3: mod_base = OTHER; note = 2&apos;-fluoronucleotide
  modified_base 7: mod_base = OTHER; note = 2&apos;-fluoronucleotide
  modified_base 9: mod_base = OTHER; note = 2&apos;-fluoronucleotide
  modified_base 2: mod_base = OTHER; note = 2&apos;-O-methylnucleotide
  modified_base 4..6: mod_base = OTHER; note = 2&apos;-O-methylnucleotide
  modified_base 8: mod_base = OTHER; note = 2&apos;-O-methylnucleotide
  modified_base 12..13: mod_base = OTHER; note = 2&apos;-O-methylnucleotide
  modified_base 11: mod_base = OTHER; note = 2&apos;-deoxythymidine
  modified_base 10: mod_base = OTHER; note = 2&apos;-fluoronucleotide
  modified_base 14: mod_base = OTHER; note = 2&apos;-fluoronucleotide
  modified_base 15..21: mod_base = OTHER; note = 2&apos;-O-methylnucleotide
  modified_base 21: mod_base = OTHER; note = Conjugated with a reversed abasic linker
ctagacctgttttgcttttgt

SEQ ID NO: 45 - RNA, 25 nt, synthetic construct
  source 1..25: mol_type = other RNA
  modified_base 1^2: mod_base = OTHER; note = Phosphorothioate Linkage
  modified_base 2^3: mod_base = OTHER; note = Phosphorothioate Linkage
  modified_base 21^22: mod_base = OTHER; note = Phosphorothioate Linkage
  modified_base 22^23: mod_base = OTHER; note = Phosphorothioate Linkage
  modified_base 2: mod_base = OTHER; note = 2&apos;-fluoronucleotide
  modified_base 4..5: mod_base = OTHER; note = 2&apos;-fluoronucleotide
  modified_base 8: mod_base = OTHER; note = 2&apos;-fluoronucleotide
  modified_base 10: mod_base = OTHER; note = 2&apos;-fluoronucleotide
  modified_base 12: mod_base = OTHER; note = 2&apos;-fluoronucleotide
  modified_base 14: mod_base = OTHER; note = 2&apos;-fluoronucleotide
  modified_base 16: mod_base = OTHER; note = 2&apos;-fluoronucleotide
  modified_base 18: mod_base = OTHER; note = 2&apos;-fluoronucleotide
  modified_base 1: mod_base = OTHER; note = 2&apos;-fluoronucleotide
  modified_base 3: mod_base = OTHER; note = 2&apos;-O-methylnucleotide
  modified_base 7: mod_base = OTHER; note = 2&apos;-O-methylnucleotide
  modified_base 9: mod_base = OTHER; note = 2&apos;-O-methylnucleotide
  modified_base 11: mod_base = OTHER; note = 2&apos;-O-methylnucleotide
  modified_base 13: mod_base = OTHER; note = 2&apos;-O-methylnucleotide
  modified_base 15: mod_base = OTHER; note = 2&apos;-O-methylnucleotide
  modified_base 17: mod_base = OTHER; note = 2&apos;-O-methylnucleotide
  modified_base 19..23: mod_base = OTHER; note = 2&apos;-O-methylnucleotide
  modified_base 6: mod_base = OTHER; note = 2&apos;-O-methylnucleotide
  modified_base 24..25: mod_base = OTHER; note = 2&apos;-deoxythymidine
acaaaagcaaaacaggtctagaatt

SEQ ID NO: 46 - RNA, 25 nt, synthetic construct
  source 1..25: mol_type = other RNA
  modified_base 1^2: mod_base = OTHER; note = Phosphorothioate Linkage
  modified_base 2^3: mod_base = OTHER; note = Phosphorothioate Linkage
  modified_base 21^22: mod_base = OTHER; note = Phosphorothioate Linkage
  modified_base 22^23: mod_base = OTHER; note = Phosphorothioate Linkage
  modified_base 2: mod_base = OTHER; note = 2&apos;-fluoronucleotide
  modified_base 4..5: mod_base = OTHER; note = 2&apos;-fluoronucleotide
  modified_base 8: mod_base = OTHER; note = 2&apos;-fluoronucleotide
  modified_base 10: mod_base = OTHER; note = 2&apos;-fluoronucleotide
  modified_base 12: mod_base = OTHER; note = 2&apos;-fluoronucleotide
  modified_base 14: mod_base = OTHER; note = 2&apos;-fluoronucleotide
  modified_base 16: mod_base = OTHER; note = 2&apos;-fluoronucleotide
  modified_base 18: mod_base = OTHER; note = 2&apos;-fluoronucleotide
  modified_base 1: mod_base = OTHER; note = 2&apos;-fluoronucleotide
  modified_base 3: mod_base = OTHER; note = 2&apos;-O-methylnucleotide
  modified_base 7: mod_base = OTHER; note = 2&apos;-O-methylnucleotide
  modified_base 9: mod_base = OTHER; note = 2&apos;-O-methylnucleotide
  modified_base 11: mod_base = OTHER; note = 2&apos;-O-methylnucleotide
  modified_base 13: mod_base = OTHER; note = 2&apos;-O-methylnucleotide
  modified_base 15: mod_base = OTHER; note = 2&apos;-O-methylnucleotide
  modified_base 17: mod_base = OTHER; note = 2&apos;-O-methylnucleotide
  modified_base 19..23: mod_base = OTHER; note = 2&apos;-O-methylnucleotide
  modified_base 6: mod_base = OTHER; note = 2&apos;-O-methylnucleotide
  modified_base 24..25: mod_base = OTHER; note = 2&apos;-O-methylnucleotide
acaaaagcaaaacaggtctagaatt

SEQ ID NO: 47 - RNA, 21 nt, synthetic construct
  source 1..21: mol_type = other RNA
  modified_base 1^2: mod_base = OTHER; note = Phosphorothioate Linkage
  modified_base 2^3: mod_base = OTHER; note = Phosphorothioate Linkage
  modified_base 7: mod_base = OTHER; note = 2&apos;-fluoronucleotide
  modified_base 9: mod_base = OTHER; note = 2&apos;-fluoronucleotide
  modified_base 1..6: mod_base = OTHER; note = 2&apos;-O-methylnucleotide
  modified_base 8: mod_base = OTHER; note = 2&apos;-O-methylnucleotide
  modified_base 10..21: mod_base = OTHER; note = 2&apos;-O-methylnucleotide
ctagacctgttttgcttttgt

SEQ ID NO: 48 - RNA, 21 nt, synthetic construct
  source 1..21: mol_type = other RNA
  modified_base 1^2: mod_base = OTHER; note = Phosphorothioate Linkage
  modified_base 2^3: mod_base = OTHER; note = Phosphorothioate Linkage
  modified_base 7: mod_base = OTHER; note = 2&apos;-fluoronucleotide
  modified_base 9: mod_base = OTHER; note = 2&apos;-fluoronucleotide
  modified_base 2..6: mod_base = OTHER; note = 2&apos;-O-methylnucleotide
  modified_base 8: mod_base = OTHER; note = 2&apos;-O-methylnucleotide
  modified_base 10..21: mod_base = OTHER; note = 2&apos;-O-methylnucleotide
  modified_base 1: mod_base = OTHER; note = 2&apos;-fluoronucleotide
ctagacctgttttgcttttgt

SEQ ID NO: 49 - RNA, 21 nt, synthetic construct
  source 1..21: mol_type = other RNA
  modified_base 1^2: mod_base = OTHER; note = Phosphorothioate Linkage
  modified_base 2^3: mod_base = OTHER; note = Phosphorothioate Linkage
  modified_base 7: mod_base = OTHER; note = 2&apos;-fluoronucleotide
  modified_base 9: mod_base = OTHER; note = 2&apos;-fluoronucleotide
  modified_base 1..2: mod_base = OTHER; note = 2&apos;-O-methylnucleotide
  modified_base 8: mod_base = OTHER; note = 2&apos;-O-methylnucleotide
  modified_base 10..21: mod_base = OTHER; note = 2&apos;-O-methylnucleotide
  modified_base 3: mod_base = OTHER; note = 2&apos;-fluoronucleotide
  modified_base 4..6: mod_base = OTHER; note = 2&apos;-O-methylnucleotide
ctagacctgttttgcttttgt

(19) United States
(12) Patent Application Publication
(10) Pub. No.: US 2026/0139255 A1
(43) Pub. Date: May 21, 2026

(54) RNAi Agent Targeting PCSK9 and Use Thereof

(71) Applicant: SYNERK BIOTECH LIMITED, Central, Hong Kong (CN)

(72) Inventors: Tao LAN, Suzhou City (CN); Weiwen JIANG, Suzhou City (CN); Dong YU, Suzhou City (CN)

(21) Appl. No.: 19/393,642

(22) Filed: Nov. 19, 2025

(30) Foreign Application Priority Data
Nov. 19, 2024 (CN) 202411659027.1

(51) Int. Cl. C12N 15/113

(52) U.S. Cl. CPC C12N 15/1137; C12Y 304/21061; C12N 2310/14; C12N 2310/315; C12N 2310/321; C12N 2310/322; C12N 2310/351

(57) ABSTRACT

The present disclosure is related to biomedicine, and specifically relates to a PCSK9-targeting RNAi agent and use thereof. The present disclosure significantly enhances the inhibitory effect of RNAi agents on PCSK9 by optimizing the modification method, thereby offering improved application potential for the prevention or treatment of PCSK9-related diseases.

### CROSS-REFERENCE TO RELATED APPLICATIONS

The present disclosure claims priority to Chinese Patent Application No. 2024116590271, filed on Nov. 19, 2024, which is incorporated herein by reference in its entirety.

### REFERENCE TO THE ELECTRONIC SEQUENCE LISTING

The contents of the electronic sequence listing (PD250287US-sequence list.xml; Size: 404,336 bytes; and Date of Creation: Nov. 12, 2025) are herein incorporated by reference in its entirety.

### TECHNICAL FIELD

The present disclosure is related to biomedicine, and specifically relates to a PCSK9-targeting RNAi agent and use thereof.

### BACKGROUND

Proprotein Convertase Subtilisin/Kexin type 9 (PCSK9) is a protein convertase that plays a critical role in the liver. It regulates blood levels of low-density lipoprotein (LDL) cholesterol by binding to LDL receptors and promoting their degradation. Overexpression of PCSK9 leads to a reduction in LDL receptors, resulting in elevated LDL cholesterol levels in the bloodstream and an increased risk of cardiovascular diseases such as hypercholesterolemia, atherosclerosis, coronary artery disease, and stroke. Therefore, PCSK9 inhibitors have become important therapeutic targets for the treatment and prevention of these conditions.

PCSK9 inhibitors affect the function and quantity of PCSK9 in the body through various mechanisms, including blocking the interaction between PCSK9 and LDL receptors (LDLR), interfering with PCSK9 secretion, and suppressing PCSK9 expression. Currently, four PCSK9 inhibitory therapies (PCSK9-iT) have been approved for clinical use: monoclonal antibodies (Evolocumab, Alirocumab, and Tafolecimab) and small interfering RNA (siRNA) therapy (Inclisiran). These treatments can significantly reduce LDL-C levels, achieving an additional 50-60% reduction compared to using statin monotherapy alone. Among them, RNAi drugs (siRNA) offer the advantage of longer-lasting efficacy compared to antibodies. Therefore, developing siRNA agents targeting PCSK9 is of great significance. However, the current siRNA therapies still have room for improvement in their inhibitory effects on PCSK9.

### SUMMARY OF THE INVENTION

A first objective of the present disclosure is to provide an RNAi agent, including a sense strand and an antisense strand, where the antisense strand includes a nucleotide sequence from positions 1 to 21 of 5′-ACAAAAGCAAAACAGGUCUAGAA-3′ (SEQ ID No. 1); the sense strand includes a nucleotide sequence that is at least partially complementary to the antisense strand; and all nucleotides in the antisense strand are modified nucleotides and meet at least one of the following conditions I) and II): I) the antisense strand does not include 3 consecutive identical chemical modifications at nucleotide positions 1 to 20 of the sequence as shown in SEQ ID No. 1; and II) the antisense strand includes 2′-fluoronucleotide at nucleotide position 1 of the sequence as shown in SEQ ID No. 1.

The present disclosure further provides a cell including the RNAi agent.

The present disclosure further provides a pharmaceutical composition including the RNAi agent.

A second objective of the present disclosure is to provide a method of inhibiting expression of PCSK9 in a cell, where the method includes: contacting the cell with the RNAi agent or the pharmaceutical composition described herein, to inhibit the expression of PCSK9 in the cell.

The present disclosure further provides the use of the RNAi agent or the pharmaceutical composition in treatment and/or prevention of a disease associated with PCSK9.

The present disclosure further provides the use of the RNAi agent or the pharmaceutical composition in preparation of a medicament for treatment and/or prevention of a disease associated with PCSK9.

The present disclosure significantly enhances the inhibitory effect of RNAi agents on PCSK9 by optimizing the modification method, thereby offering improved therapeutic potential for the prevention or treatment of PCSK9-related diseases.

### DETAILED DESCRIPTION

The specific embodiments of the present disclosure are described in detail hereinafter. It should be understood that the specific embodiments described herein are only intended to illustrate and explain the present disclosure, rather than limiting the present disclosure. A person skilled in the art can make various modifications and changes to the present disclosure without departing from the scope or spirit of the present disclosure. For example, features illustrated or described as a part of an embodiment can be used in another embodiment to create still another embodiment.

Unless otherwise specified, all terms (including technical and scientific terms) used to disclose the present disclosure have the same meanings as commonly understood by a person of ordinary skill in the art to which the present disclosure belongs. With reference to further guidance, the following definitions are used to better understand teachings in the present disclosure. The terms used herein in the description of the present disclosure are only for the purpose of describing specific examples, and are not intended to limit the present disclosure.

The term “and/or” as used herein refers to a selection range that includes any one of the listed items among two or more related items, as well as any and all combinations of those listed items. These combinations may include any two of the related items, any greater number of the related items, or all of the related items. In the present disclosure, it should be understood that when at least two items are connected by “and/or”, the technical solutions undoubtedly include technical solutions connected by logical “AND”, and also undoubtedly include technical solutions connected by logical “OR”. For example, “A and/or B” includes three parallel solutions: A, B, and both A and B. For another example, technical solutions of “A, B, C and/or D” include any one of A, B, C and D (namely, a technical solution connected by logical “OR”), and also include a combination of any one and all of A, B, C and D, that is, a combination of any two or three of A, B, C and D and a combination of four of A, B, C and D (namely, technical solutions connected by logical “AND”).

The terms “comprise”, “include”, and “contain”, as well as their variations, as used herein are synonymous, and are inclusive or open-ended, and do not exclude additional uncited members, elements, or method steps.

In the present disclosure, a numerical range defined by endpoints includes all values and fractions within the range, as well as the cited endpoints.

In the present disclosure, concentration values refer to values that may fluctuate within a certain range. For example, fluctuations may occur within the corresponding precision range. For instance, a value of 2% may allow fluctuations within ±0.1%. For larger values or values that do not require precise control, the meaning may include greater fluctuations. For example, a value of 100 mM may allow fluctuations within ±1%, 2%, ±5%, etc. For molecular weight, the meaning may include fluctuations within ±10%.

In the present disclosure, descriptions such as “multiple” and “various” refer to two or more than two, unless otherwise specified.

In the present disclosure, technical features described in an open-ended manner encompass both closed technical solutions consisting of the listed features and open technical solutions that include the listed features.

In the present disclosure, expressions such as “preferably”, “more preferably”, “most preferably”, or “desirably” are used solely to describe embodiments or examples with better effects, and shall not be construed as limiting the scope of protection of the invention.

In the present disclosure, terms such as “optionally”, “optional”, or equivalents thereof indicate that the feature may or may not be present, i.e., selected from two parallel options: presence or absence. Where multiple instances of “optional” features appear in a technical solution, unless otherwise specified and provided there is no contradiction or mutual exclusivity, each “optional” feature shall be considered independently.

In the present disclosure, the term “RNAi agent” (also referred to as “RNAi trigger”) refers to a composition containing RNA or RNA-like (e.g., chemically modified RNA) oligonucleotide molecules, and the oligonucleotide molecules are capable of degrading or inhibiting (e.g., degrading or inhibiting under proper conditions) translation of messenger RNA (mRNA) transcripts of the target mRNA in a sequence-specific manner. As used herein, the RNAi agent can exert an effect through the RNA interference mechanism (that is, RNA interference induced by interaction via an RNA interference pathway mechanism (RNA-induced silencing complex, RISC) of mammalian cells) or any alternative mechanism or pathway. Although it is believed that the RNAi agent, as the term is used herein, exerts an effect primarily via the RNA interference mechanism, the disclosed RNAi agent is not bound or limited by any specific pathway or mechanism of action. The RNAi agent disclosed herein is composed of a sense strand and an antisense strand, and includes, but is not limited to, short (or small) interfering RNA (siRNA), double-stranded RNA (dsRNA), microRNA (miRNA), short hairpin RNA (shRNA), and dicer substrates. The antisense strand of the RNAi agent described herein is at least partially complementary to the to-be-targeted mRNA (that is, PCSK9 mRNA). The RNAi agent may include one or more modified nucleotides and/or one or more non-phosphodiester linkages.

In the present disclosure, when referring to the expression of a given gene, the term “silencing”, “reduction”, “inhibition”, “downregulation” or “knockdown” refers to a decrease in gene expression, as measured by the level of RNA transcribed from the gene or the level of polypeptide, protein, or protein subunit translated from the mRNA, in cells, cell populations, tissues, organs, or subjects in which the gene is transcribed, when treated with the RNAi agent described herein, as compared to untreated cells, cell populations, tissues, organs, or subjects.

In the present disclosure, the term “fully complementary” means that, in a hybridized pair of nucleobases or nucleotide sequence molecules, all (100%) of the bases in a contiguous sequence of the first oligonucleotide hybridize with the same number of bases in a contiguous sequence of the second oligonucleotide. The contiguous sequence may include all or part of the first nucleotide sequence or the second nucleotide sequence.

In the present disclosure, the term “partially complementary” means that, in a hybridized pair of nucleobases or nucleotide sequence molecules, at least 70%, but not all, of the bases in a contiguous sequence of the first oligonucleotide hybridize with the same number of bases in a contiguous sequence of the second oligonucleotide. The contiguous sequence may include all or part of the first nucleotide sequence or the second nucleotide sequence.

In the present disclosure, the term “substantially complementary” means that, in a hybridized pair of nucleobases or nucleotide sequence molecules, at least 85%, but not all, of the bases in a contiguous sequence of the first oligonucleotide hybridize with the same number of bases in a contiguous sequence of the second oligonucleotide. The contiguous sequence may include all or part of the first nucleotide sequence or the second nucleotide sequence.

In the present disclosure, the term “at least partially complementary” means that, in a hybridized pair of nucleobases or nucleotide sequence molecules, the first oligonucleotide and the second oligonucleotide are partially, substantially, or fully complementary.

In the present disclosure, the term “treatment” refers to a method or step taken to relieve or alleviate the number, severity, and/or frequency of one or more disease symptoms in a subject. The treatment may include the prevention, management, prophylactic treatment, and/or inhibition or reduction of the number, severity, and/or frequency of one or more disease symptoms in a subject.

In the present disclosure, the term “link” means that two compounds or molecules are joined through a covalent bond. Unless otherwise specified, as used herein, the term “link” may refer to a connection between a first compound and a second compound with or without any intermediate atom or group of atoms.

In the present disclosure, the phrase “the nucleotide position A of the sequence as shown in SEQ ID No. B” refers to a position relative to the specific sequence. When the nucleotide position in the sequence is altered (e.g., due to the addition of a nucleotide at the 5′ end), a person skilled in the art can still identify the corresponding nucleotide position in the specific sequence based on the nucleotide that corresponds to the position defined in the present disclosure, and such identification shall also fall within the scope of protection of the present disclosure.

In the present disclosure, the letters ‘G’, ‘C’, ‘A’, and ‘U’ usually represent nucleotides including guanine, cytosine, adenine, and uracil bases, respectively. ‘T’ and ‘dT’ are used interchangeably, and specifically refer to deoxyribonucleotides with a base of thymine (e.g., deoxyribosylthymine, 2′-deoxythymidine, or thymidine). It should be noted that the term “ribonucleotide”, “nucleotide”, or “deoxyribonucleotide” also encompasses a modified nucleotide (for details, refer to the following description) or an alternative substitution group thereof. It is well known to a person skilled in the art that when guanine, cytosine, adenine, or uracil is replaced with another group, the overall function of the oligonucleotide remains substantially unaffected, provided that the substitution group contained in the nucleotide maintains a base-pairing ability. For example (including, but not limited to), a nucleotide containing an inosine base (I) can pair with a nucleotide containing adenine, cytosine, or uracil. Therefore, the uracil, guanine, or adenine nucleotide in the nucleotide sequence in the present disclosure can be replaced with a nucleotide containing a substitution group such as inosine. All such sequences with alternative bases shall fall within the protection scope of the present disclosure.

In the present disclosure, the “modified nucleotide” is one or more selected from 2′-O-methyl nucleotide, 2′-fluoronucleotide, 2′-deoxy nucleotide, 2′,3′-seco nucleotide mimetic, locked nucleotide, 2′-F-arabinonucleotide, 2′-methoxyethyl nucleotide, abasic nucleotide, ribitol, inverted nucleotide, inverted 2′-O-methyl nucleotide, inverted 2′-deoxy nucleotide, 2′-amino-modified nucleotide, 2′-alkyl-modified nucleotide, morpholino nucleotide, peptide nucleic acid, glycerol nucleic acid, triazole-linked DNA, nucleotide containing an unnatural base, nucleotide containing vinyl phosphonate, nucleotide containing cyclopropyl phosphonate, and 3′-O-methyl nucleotide. In some specific embodiments, the modified nucleotide is selected from 2′-O-methyl nucleotide, 2′-fluoronucleotide, and 2′-deoxy nucleotide.

In the present disclosure, the “chemical modification” includes but is not limited to the following modifications: 2′-O-methyl modification, 2′-fluoro modification, 2′-deoxy modification, 2′-F-arabinose modification, 2′-methoxyethyl modification, 2′-amino modification, 2′-alkyl modification, 3′-O-methyl modification, locked nucleic acid methylene bridge modification, abasic modification, unnatural base modification, vinyl phosphonate modification, and cyclopropyl phosphonate modification. In some specific embodiments, the chemical modification is selected from 2′-O-methyl modification and 2′-fluoro modification.

### RNAi Agent

A first objective of the present disclosure is to provide an RNAi agent, including a sense strand and an antisense strand, where the antisense strand includes a nucleotide sequence from positions 1 to 21 of 5′-ACAAAAGCAAAACAGGUCUAGAA-3′ (SEQ ID No. 1); the sense strand includes a nucleotide sequence that is at least partially complementary (e.g., partially complementary, substantially complementary, or fully complementary) to the antisense strand; and all nucleotides in the antisense strand are modified nucleotides and meet at least one of the following conditions I) and II): I) the antisense strand does not include 3 consecutive identical chemical modifications at nucleotide positions 1 to 20 of the sequence as shown in SEQ ID No. 1; and II) the antisense strand includes 2′-fluoronucleotide at nucleotide position 1 of the sequence as shown in SEQ ID No. 1. As discovered in the present disclosure, applying the aforementioned modifications to the antisense strand significantly enhances the inhibitory effect of the RNAi agent on PCSK9.

In some embodiments, the antisense strand meets the condition I) as described above.

In some embodiments, the antisense strand meets the condition II) as described above.

In some embodiments, the antisense strand meets both the conditions I) and II) as described above.

In some embodiments, modified nucleotides at the remaining positions in the antisense strand are each independently selected from 2′-O-methyl nucleotide and 2′-fluoronucleotide.

In some embodiments, the antisense strand includes a nucleotide sequence in 5′-ACAAAAGCAAAACAGGUCUAGAA-3′ (SEQ ID No. 1).

In some embodiments, the antisense strand includes 2′-fluoronucleotide at nucleotide positions 4 and 5 and 2′-O-methyl nucleotide at nucleotide position 6 of the sequence as shown in SEQ ID No. 1. This modification exhibits significantly improved inhibitory effect on PCSK9, with a more pronounced advantage observed in in vivo experiments, compared to the case where all three positions are 2′-fluoro nucleotides.

In some embodiments, the antisense strand includes 2′-fluoronucleotide at nucleotide positions 2 and 14 of the sequence as shown in SEQ ID No. 1.

In some embodiments, the antisense strand includes 2′-fluoronucleotide at nucleotide positions 2, 8, 10, 12, 14, 16, and 18 of the sequence as shown in SEQ ID No. 1.

In some embodiments, the sense strand includes a nucleotide sequence of 5′-CUAGACCUGUUUUGCUUUUGU-3′ (SEQ ID No.2) or 5′-CUAGACCUGUTUUGCUUUUGU-3′ (SEQ ID No.3).

In some preferred embodiments, all nucleotides in the sense strand are modified nucleotides; the sense strand includes 2′-fluoronucleotide at nucleotide positions 1 and/or 3 of the sequence as shown in SEQ ID No. 2 or 3; and wherein, when the sense strand comprises the nucleotide sequence shown in SEQ ID No. 3, the nucleotide at position 11 is 2′-deoxythymidine. Such modification of the sense strand can further improve the inhibitory effect of the RNAi agent on PCSK9.

In some specific embodiments, all nucleotides in the sense strand are modified nucleotides, and the sense strand includes 2′-fluoronucleotide at nucleotide position 1 of the sequence as shown in SEQ ID No. 2 or 3.

In some specific embodiments, all nucleotides in the sense strand are modified nucleotides, and the sense strand includes 2′-fluoronucleotide at nucleotide position 3 of the sequence as shown in SEQ ID No. 2 or 3.

In some preferred embodiments, all nucleotides in the sense strand are modified nucleotides, and the sense strand includes 2′-fluoronucleotide at nucleotide positions 1 and 3 of the sequence as shown in SEQ ID No. 2 or 3.

In some embodiments, modified nucleotides at the remaining positions in the sense strand are each independently selected from 2′-O-methyl nucleotide and 2′-fluoronucleotide.

In some embodiments, the sense strand includes the nucleotide sequence of 5′-CUAGACCUGUUUUGCUUUUGU-3′ (SEQ ID No. 2), and the sense strand includes 2′-fluoronucleotide at nucleotide position 11 of the sequence as shown in SEQ ID No. 2. As discovered in the present disclosure, when nucleotide position 11 of the sequence as shown in SEQ ID No. 2 is 2′-fluoronucleotide, the inhibitory effect of the RNAi agent on PCSK9 significantly improves compared to the case where this position is 2′-deoxythymidinein in the sequence as shown in SEQ ID No.3.

In some embodiments, the sense strand includes 2′-fluoronucleotide at nucleotide position 10 of the sequence as shown in SEQ ID No. 2 or 3. As discovered in the present disclosure, this modification further improves the inhibitory effect of the RNAi agent on PCSK9.

In a preferred embodiment, the sense strand includes the nucleotide sequence of 5′-CUAGACCUGUUUUGCUUUUGU-3′ (SEQ ID No. 2), and the sense strand includes 2′-fluoronucleotide at nucleotide positions 10 and 11 of the sequence as shown in SEQ ID No. 2. Such a sequence and modification method exhibits a significantly superior inhibitory effect on PCSK9 compared to the case where 2′-O-methyl nucleotide and 2′-fluoronucleotide are sequentially positioned at the corresponding sites.

In some embodiments, the sense strand includes 2′-fluoronucleotide at nucleotide positions 7 and/or 9 of the sequence as shown in SEQ ID No. 2 or 3.

In some embodiments, the nucleotides of the sense strand at nucleotide positions 8 and 14 of the sequence as shown in SEQ ID No. 2 or 3 are each independently selected from 2′-fluoronucleotide and 2′-O-methyl nucleotide, and modified nucleotides at the remaining positions are 2′-O-methyl nucleotides.

In some embodiments, at least one (e.g., one or two) phosphorothioate internucleotide linkage is present among the last three nucleotides at the 5′ end of the sense strand.

In some specific embodiments, two phosphorothioate internucleotide linkages are present among the last three nucleotides at the 5′ end of the sense strand. In some specific embodiments, one phosphorothioate internucleotide linkage is present between the last two nucleotides at the 5′ end of the sense strand. In some specific embodiments, one phosphorothioate internucleotide linkage is present between the third and second nucleotides from the 5′ end of the sense strand.

In some embodiments, at least one (e.g., one or two) phosphorothioate internucleotide linkage is present among the last three nucleotides at the 5′ end of the antisense strand, and at least one (e.g., one, two, three, or four) phosphorothioate internucleotide linkage is present among the last five nucleotides at the 3′ end of the antisense strand.

In some specific embodiments, two phosphorothioate internucleotide linkages are present among the last three nucleotides at the 5′ end of the antisense strand. In some specific embodiments, one phosphorothioate internucleotide linkage is present between the last two nucleotides at the 5′ end of the antisense strand. In yet other specific embodiments, one phosphorothioate internucleotide linkage is present between the third and second nucleotides from the 5′ end of the antisense strand.

In some specific embodiments, two phosphorothioate internucleotide linkages are present among the last three nucleotides at the 3′ end of the antisense strand. In some specific embodiments, two phosphorothioate internucleotide linkages are present among the fourth to second nucleotides from the 3′ end of the antisense strand. In some specific embodiments, two phosphorothioate internucleotide linkages are present among the fifth to third nucleotides from the 3′ end of the antisense strand. In some specific embodiments, one phosphorothioate internucleotide linkage is present between the last two nucleotides at the 3′ end of the antisense strand. In some specific embodiments, one phosphorothioate internucleotide linkage is present between the third and second nucleotides from the 3′ end of the antisense strand. In some specific embodiments, one phosphorothioate internucleotide linkage is present between the fourth and third nucleotides from the 3′ end of the antisense strand. In some specific embodiments, one phosphorothioate internucleotide linkage is present between the fifth and fourth nucleotides from the 3′ end of the antisense strand.

In some embodiments, the 5′ end and/or 3′ end of the sense strand contains an inverted abasic linker or reversed abasic linker.

In some embodiments, the 3′ end of the antisense strand contains at least one (e.g., one, two, or three) 2′-deoxythymidine.

In some embodiments, the 3′ end of the antisense strand contains at least one (e.g., one, two, or three) 2′-O-methyluridine.

A person skilled in the art can combine the foregoing embodiments with reference to common knowledge, to obtain more embodiments of the RNAi agent in the present disclosure.

In some specific embodiments, the antisense strands and sense strands as described above can be combined to obtain RNAi agents (siRNAs) with sense strand and antisense strand sequences shown in Table 1.

TABLE 1

| Name of | | | | |
|---|---|---|---|---|
| siRNA | Sense strand (5′-3′) | SEQ ID No | Antisense strand (5′-3′) | SEQ ID No. |
| SNK-0008 | CfsusAfgacCfuGfUfUfuugcuuuugu | 4 | AfsCfsaAfAfAfgCfaAfaAfcAfgGfuCfuagsasa | 12 |
| SNK-0009 | CfsusAfgacCfuGfUfUfuuGfcuuuugu | 5 | AfsCfsaAfAfAfgCfaAfaAfcAfgGfuCfuagsasa | 12 |
| SNK-0010 | CfsusAfgacCfUfGfUfUfuuGfcuuuugu | 6 | AfsCfsaAfAfAfgCfaAfaAfcAfgGfuCfuagsasa | 12 |
| SNK-0011 | CfsusAfgacCfuGfudTuugcuuuugu | 7 | AfsCfsaAfAfAfgCfaAfaAfcAfgGfuCfuagsasa | 12 |
| SNK-0012 | CfsusAfgacCfuGfudTuuGfcuuuugu | 8 | AfsCfsaAfAfAfgCfaAfaAfcAfgGfuCfuagsasa | 12 |
| SNK-0013 | CfsusAfgacCfUfGfudTuuGfcuuuugu | 9 | AfsCfsaAfAfAfgCfaAfaAfcAfgGfuCfuagsasa | 12 |
| SNK-0014 | CfsusAfgacCfuGfUfdTuuGfcuuuugu | 10 | AfsCfsaAfAfAfgCfaAfaAfcAfgGfuCfuagsasa | 12 |
| SNK-0015 | csusagacCfuGfudTuugcuuuugu | 14 | AfsCfsaAfAfAfgCfaAfaAfcAfgGfuCfuagsasa | 12 |
| SNK-0016 | CfsusAfgacCfuGfUfUfuugcuuuugu | 4 | AfsCfsaAfAfagCfaAfaAfcAfgGfuCfuagsasa | 13 |
| SNK-0017 | CfsusAfgacCfuGfUfUfuuGfcuuuugu | 5 | AfsCfsaAfAfagCfaAfaAfcAfgGfuCfuagsasa | 13 |
| SNK-0018 | CfsusAfgacCfUfGfUfUfuuGfcuuuugu | 6 | AfsCfsaAfAfagCfaAfaAfcAfgGfuCfuagsasa | 13 |
| SNK-0019 | CfsusAfgacCfuGfudTuugcuuuugu | 7 | AfsCfsaAfAfagCfaAfaAfcAfgGfuCfuagsasa | 13 |
| SNK-0020 | CfsusAfgacCfuGfudTuuGfcuuuugu | 8 | AfsCfsaAfAfagCfaAfaAfcAfgGfuCfuagsasa | 13 |
| SNK-0021 | CfsusAfgacCfUfGfudTuuGfcuuuugu | 9 | AfsCfsaAfAfagCfaAfaAfcAfgGfuCfuagsasa | 13 |
| SNK-0022 | CfsusAfgacCfuGfUfdTuuGfcuuuugu | 10 | AfsCfsaAfAfagCfaAfaAfcAfgGfuCfuagsasa | 13 |
| SNK-0023 | csusagacCfuGfudTuugcuuuugu | 14 | AfsCfsaAfAfagCfaAfaAfcAfgGfuCfuagsasa | 13 |
| SNK-0024 | CfsuAfgacCfuGfUfUfuugcuuuugu | 15 | AfsCfsaAfAfAfgCfaAfaAfcAfgGfuCfuagsasa | 12 |
| SNK-0025 | CfsusAfgacCfuGfUfUfuugcuuuugu | 4 | AfsCfsaAfAfAfgCfaAfaAfcAfgGfuCfuagsasadTdT | 16 |
| SNK-0026 | CfsusAfgacCfuGfUfUfuugcuuuugu | 4 | AfsCfsaAfAfAfgCfaAfaAfcAfgGfuCfuagsasauu | 17 |
| SNK-0027 | RalCfsusAfgacCfuGfUfUfuugcuuuugu | 18 | AfsCfsaAfAfAfgCfaAfaAfcAfgGfuCfuagsasa | 12 |
| SNK-0028 | RalCfsusAfgacCfuGfUfUfuugcuuuuguRal | 19 | AfsCfsaAfAfAfgCfaAfaAfcAfgGfuCfuagsasa | 12 |
| SNK-0029 | CfsusAfgacCfuGfUfUfuugcuuuuguRal | 20 | AfsCfsaAfAfAfgCfaAfaAfcAfgGfuCfuagsasa | 12 |
| SNK-0030 | CfsuAfgacCfuGfUfUfuuGfcuuuugu | 21 | AfsCfsaAfAfAfgCfaAfaAfcAfgGfuCfuagsasa | 12 |
| SNK-0031 | CfsusAfgacCfuGfUfUfuuGfcuuuugu | 5 | AfsCfsaAfAfAfgCfaAfaAfcAfgGfuCfuagsasadTdT | 16 |
| SNK-0032 | CfsusAfgacCfuGfUfUfuuGfcuuuugu | 5 | AfsCfsaAfAfAfgCfaAfaAfcAfgGfuCfuagsasauu | 17 |
| SNK-0033 | RalCfsusAfgacCfuGfUfUfuuGfcuuuugu | 22 | AfsCfsaAfAfAfgCfaAfaAfcAfgGfuCfuagsasa | 12 |
| SNK-0034 | RalCfsusAfgacCfuGfUfUfuuGfcuuuuguRal | 23 | AfsCfsaAfAfAfgCfaAfaAfcAfgGfuCfuagsasa | 12 |
| SNK-0035 | CfsusAfgacCfuGfUfUfuuGfcuuuuguRal | 24 | AfsCfsaAfAfAfgCfaAfaAfcAfgGfuCfuagsasa | 12 |
| SNK-0036 | CfsuAfgacCfUfGfUfUfuuGfcuuuugu | 25 | AfsCfsaAfAfAfgCfaAfaAfcAfgGfuCfuagsasa | 12 |
| SNK-0037 | CfsusAfgacCfUfGfUfUfuuGfcuuuugu | 6 | AfsCfsaAfAfAfgCfaAfaAfcAfgGfuCfuagsasadTdT | 16 |
| SNK-0038 | CfsusAfgacCfUfGfUfUfuuGfcuuuugu | 6 | AfsCfsaAfAfAfgCfaAfaAfcAfgGfuCfuagsasauu | 17 |
| SNK-0039 | RalCfsusAfgacCfUfGfUfUfuuGfcuuuugu | 26 | AfsCfsaAfAfAfgCfaAfaAfcAfgGfuCfuagsasa | 12 |
| SNK-0040 | RalCfsusAfgacCfUfGfUfUfuuGfcuuuuguRal | 27 | AfsCfsaAfAfAfgCfaAfaAfcAfgGfuCfuagsasa | 12 |
| SNK-0041 | CfsusAfgacCfUfGfUfUfuuGfcuuuuguRal | 28 | AfsCfsaAfAfAfgCfaAfaAfcAfgGfuCfuagsasa | 12 |
| SNK-0042 | CfsuAfgacCfuGfudTuugcuuuugu | 29 | AfsCfsaAfAfAfgCfaAfaAfcAfgGfuCfuagsasa | 12 |
| SNK-0043 | CfsusAfgacCfuGfudTuugcuuuugu | 7 | AfsCfsaAfAfAfgCfaAfaAfcAfgGfuCfuagsasadTdT | 16 |
| SNK-0044 | CfsusAfgacCfuGfudTuugcuuuugu | 7 | AfsCfsaAfAfAfgCfaAfaAfcAfgGfuCfuagsasauu | 17 |
| SNK-0045 | RalCfsusAfgacCfuGfudTuugcuuuugu | 30 | AfsCfsaAfAfAfgCfaAfaAfcAfgGfuCfuagsasa | 12 |
| SNK-0046 | RalCfsusAfgacCfuGfudTuugcuuuuguRal | 31 | AfsCfsaAfAfAfgCfaAfaAfcAfgGfuCfuagsasa | 12 |
| SNK-0047 | CfsusAfgacCfuGfudTuugcuuuuguRal | 32 | AfsCfsaAfAfAfgCfaAfaAfcAfgGfuCfuagsasa | 12 |
| SNK-0048 | CfsuAfgacCfuGfudTuuGfcuuuugu | 33 | AfsCfsaAfAfAfgCfaAfaAfcAfgGfuCfuagsasa | 12 |
| SNK-0049 | CfsusAfgacCfuGfudTuuGfcuuuugu | 8 | AfsCfsaAfAfAfgCfaAfaAfcAfgGfuCfuagsasadTdT | 16 |
| SNK-0050 | CfsusAfgacCfuGfudTuuGfcuuuugu | 8 | AfsCfsaAfAfAfgCfaAfaAfcAfgGfuCfuagsasauu | 17 |
| SNK-0051 | RalCfsusAfgacCfuGfudTuuGfcuuuugu | 34 | AfsCfsaAfAfAfgCfaAfaAfcAfgGfuCfuagsasa | 12 |
| SNK-0052 | RalCfsusAfgacCfuGfudTuuGfcuuuuguRal | 35 | AfsCfsaAfAfAfgCfaAfaAfcAfgGfuCfuagsasa | 12 |
| SNK-0053 | CfsusAfgacCfuGfudTuuGfcuuuuguRal | 36 | AfsCfsaAfAfAfgCfaAfaAfcAfgGfuCfuagsasa | 12 |
| SNK-0054 | CfsuAfgacCfUfGfudTuuGfcuuuugu | 37 | AfsCfsaAfAfAfgCfaAfaAfcAfgGfuCfuagsasa | 12 |
| SNK-0055 | CfsusAfgacCfUfGfudTuuGfcuuuugu | 9 | AfsCfsaAfAfAfgCfaAfaAfcAfgGfuCfuagsasadTdT | 16 |
| SNK-0056 | CfsusAfgacCfUfGfudTuuGfcuuuugu | 9 | AfsCfsaAfAfAfgCfaAfaAfcAfgGfuCfuagsasauu | 17 |
| SNK-0057 | RalCfsusAfgacCfUfGfudTuuGfcuuuugu | 38 | AfsCfsaAfAfAfgCfaAfaAfcAfgGfuCfuagsasa | 12 |
| SNK-0058 | RalCfsusAfgacCfUfGfudTuuGfcuuuuguRal | 39 | AfsCfsaAfAfAfgCfaAfaAfcAfgGfuCfuagsasa | 12 |
| SNK-0059 | CfsusAfgacCfUfGfudTuuGfcuuuuguRal | 40 | AfsCfsaAfAfAfgCfaAfaAfcAfgGfuCfuagsasa | 12 |
| SNK-0060 | CfsuAfgacCfuGfUfdTuuGfcuuuugu | 41 | AfsCfsaAfAfAfgCfaAfaAfcAfgGfuCfuagsasa | 12 |
| SNK-0061 | CfsusAfgacCfuGfUfdTuuGfcuuuugu | 10 | AfsCfsaAfAfAfgCfaAfaAfcAfgGfuCfuagsasadTdT | 16 |
| SNK-0062 | CfsusAfgacCfuGfUfdTuuGfcuuuugu | 10 | AfsCfsaAfAfAfgCfaAfaAfcAfgGfuCfuagsasauu | 17 |
| SNK-0063 | RalCfsusAfgacCfuGfUfdTuuGfcuuuugu | 42 | AfsCfsaAfAfAfgCfaAfaAfcAfgGfuCfuagsasa | 12 |
| SNK-0064 | RalCfsusAfgacCfuGfUfdTuuGfcuuuuguRal | 43 | AfsCfsaAfAfAfgCfaAfaAfcAfgGfuCfuagsasa | 12 |
| SNK-0065 | CfsusAfgacCfuGfUfdTuuGfcuuuuguRal | 44 | AfsCfsaAfAfAfgCfaAfaAfcAfgGfuCfuagsasa | 12 |
| SNK-0066 | CfsuAfgacCfuGfUfUfuugcuuuugu | 15 | AfsCfsaAfAfagCfaAfaAfcAfgGfuCfuagsasa | 13 |
| SNK-0067 | CfsusAfgacCfuGfUfUfuugcuuuugu | 4 | AfsCfsaAfAfagCfaAfaAfcAfgGfuCfuagsasadTdT | 45 |
| SNK-0068 | CfsusAfgacCfuGfUfUfuugcuuuugu | 4 | AfsCfsaAfAfagCfaAfaAfcAfgGfuCfuagsasauu | 46 |
| SNK-0069 | RalCfsusAfgacCfuGfUfUfuugcuuuugu | 18 | AfsCfsaAfAfagCfaAfaAfcAfgGfuCfuagsasa | 13 |
| SNK-0070 | RalCfsusAfgacCfuGfUfUfuugcuuuuguRal | 19 | AfsCfsaAfAfagCfaAfaAfcAfgGfuCfuagsasa | 13 |
| SNK-0071 | CfsusAfgacCfuGfUfUfuugcuuuuguRal | 20 | AfsCfsaAfAfagCfaAfaAfcAfgGfuCfuagsasa | 13 |
| SNK-0072 | CfsuAfgacCfuGfUfUfuuGfcuuuugu | 21 | AfsCfsaAfAfagCfaAfaAfcAfgGfuCfuagsasa | 13 |
| SNK-0073 | CfsusAfgacCfuGfUfUfuuGfcuuuugu | 5 | AfsCfsaAfAfagCfaAfaAfcAfgGfuCfuagsasadTdT | 45 |
| SNK-0074 | CfsusAfgacCfuGfUfUfuuGfcuuuugu | 5 | AfsCfsaAfAfagCfaAfaAfcAfgGfuCfuagsasauu | 46 |
| SNK-0075 | RalCfsusAfgacCfuGfUfUfuuGfcuuuugu | 22 | AfsCfsaAfAfagCfaAfaAfcAfgGfuCfuagsasa | 13 |
| SNK-0076 | RalCfsusAfgacCfuGfUfUfuuGfcuuuuguRal | 23 | AfsCfsaAfAfagCfaAfaAfcAfgGfuCfuagsasa | 13 |
| SNK-0077 | CfsusAfgacCfuGfUfUfuuGfcuuuuguRal | 24 | AfsCfsaAfAfagCfaAfaAfcAfgGfuCfuagsasa | 13 |
| SNK-0078 | CfsuAfgacCfUfGfUfUfuuGfcuuuugu | 25 | AfsCfsaAfAfagCfaAfaAfcAfgGfuCfuagsasa | 13 |
| SNK-0079 | CfsusAfgacCfUfGfUfUfuuGfcuuuugu | 6 | AfsCfsaAfAfagCfaAfaAfcAfgGfuCfuagsasadTdT | 45 |
| SNK-0080 | CfsusAfgacCfUfGfUfUfuuGfcuuuugu | 6 | AfsCfsaAfAfagCfaAfaAfcAfgGfuCfuagsasauu | 46 |
| SNK-0081 | RalCfsusAfgacCfUfGfUfUfuuGfcuuuugu | 26 | AfsCfsaAfAfagCfaAfaAfcAfgGfuCfuagsasa | 13 |
| SNK-0082 | RalCfsusAfgacCfUfGfUfUfuuGfcuuuuguRal | 27 | AfsCfsaAfAfagCfaAfaAfcAfgGfuCfuagsasa | 13 |
| SNK-0083 | CfsusAfgacCfUfGfUfUfuuGfcuuuuguRal | 28 | AfsCfsaAfAfagCfaAfaAfcAfgGfuCfuagsasa | 13 |
| SNK-0084 | CfsuAfgacCfuGfuTuugcuuuugu | 29 | AfsCfsaAfAfagCfaAfaAfcAfgGfuCfuagsasa | 13 |
| SNK-0085 | CfsusAfgacCfuGfudTuugcuuuugu | 7 | AfsCfsaAfAfagCfaAfaAfcAfgGfuCfuagsasadTdT | 45 |
| SNK-0086 | CfsusAfgacCfuGfudTuugcuuuugu | 7 | AfsCfsaAfAfagCfaAfaAfcAfgGfuCfuagsasauu | 46 |
| SNK-0087 | RalCfsusAfgacCfuGfudTuugcuuuugu | 30 | AfsCfsaAfAfagCfaAfaAfcAfgGfuCfuagsasa | 13 |
| SNK-0088 | RalCfsusAfgacCfuGfudTuugcuuuuguRal | 31 | AfsCfsaAfAfagCfaAfaAfcAfgGfuCfuagsasa | 13 |
| SNK-0089 | CfsusAfgacCfuGfudTuugcuuuuguRal | 32 | AfsCfsaAfAfagCfaAfaAfcAfgGfuCfuagsasa | 13 |
| SNK-0090 | CfsuAfgacCfuGfudTuuGfcuuuugu | 33 | AfsCfsaAfAfagCfaAfaAfcAfgGfuCfuagsasa | 13 |
| SNK-0091 | CfsusAfgacCfuGfudTuuGfcuuuugu | 8 | AfsCfsaAfAfagCfaAfaAfcAfgGfuCfuagsasadTdT | 45 |
| SNK-0092 | CfsusAfgacCfuGfudTuuGfcuuuugu | 8 | AfsCfsaAfAfagCfaAfaAfcAfgGfuCfuagsasauu | 46 |
| SNK-0093 | RalCfsusAfgacCfuGfudTuuGfcuuuugu | 34 | AfsCfsaAfAfagCfaAfaAfcAfgGfuCfuagsasa | 13 |
| SNK-0094 | RalCfsusAfgacCfuGfudTuuGfcuuuuguRal | 35 | AfsCfsaAfAfagCfaAfaAfcAfgGfuCfuagsasa | 13 |
| SNK-0095 | CfsusAfgacCfuGfudTuuGfcuuuuguRal | 36 | AfsCfsaAfAfagCfaAfaAfcAfgGfuCfuagsasa | 13 |
| SNK-0096 | CfsuAfgacCfUfGfudTuuGfcuuuugu | 37 | AfsCfsaAfAfagCfaAfaAfcAfgGfuCfuagsasa | 13 |
| SNK-0097 | CfsusAfgacCfUfGfudTuuGfcuuuugu | 9 | AfsCfsaAfAfagCfaAfaAfcAfgGfuCfuagsasadTdT | 45 |
| SNK-0098 | CfsusAfgacCfUfGfudTuuGfcuuuugu | 9 | AfsCfsaAfAfagCfaAfaAfcAfgGfuCfuagsasauu | 46 |
| SNK-0099 | RalCfsusAfgacCfUfGfudTuuGfcuuuugu | 38 | AfsCfsaAfAfagCfaAfaAfcAfgGfuCfuagsasa | 13 |
| SNK-0100 | RalCfsusAfgacCfUfGfudTuuGfcuuuuguRal | 39 | AfsCfsaAfAfagCfaAfaAfcAfgGfuCfuagsasa | 13 |
| SNK-0101 | CfsusAfgacCfUfGfudTuuGfcuuuuguRal | 40 | AfsCfsaAfAfagCfaAfaAfcAfgGfuCfuagsasa | 13 |
| SNK-0102 | CfsuAfgacCfuGfUfdTuuGfcuuuugu | 41 | AfsCfsaAfAfagCfaAfaAfcAfgGfuCfuagsasa | 13 |
| SNK-0103 | CfsusAfgacCfuGfUfdTuuGfcuuuugu | 10 | AfsCfsaAfAfagCfaAfaAfcAfgGfuCfuagsasadTdT | 45 |
| SNK-0104 | CfsusAfgacCfuGfUfdTuuGfcuuuugu | 10 | AfsCfsaAfAfagCfaAfaAfcAfgGfuCfuagsasauu | 46 |
| SNK-0105 | RalCfsusAfgacCfuGfUfdTuuGfcuuuugu | 42 | AfsCfsaAfAfagCfaAfaAfcAfgGfuCfuagsasa | 13 |
| SNK-0106 | RalCfsusAfgacCfuGfUfdTuuGfcuuuuguRal | 43 | AfsCfsaAfAfagCfaAfaAfcAfgGfuCfuagsasa | 13 |
| SNK-0107 | CfsusAfgacCfuGfUfdTuuGfcuuuuguRal | 44 | AfsCfsaAfAfagCfaAfaAfcAfgGfuCfuagsasa | 13 |
| SNK-0108 | csusagacCfuGfuuuugcuuuugu | 47 | AfsCfsaAfAfAfgCfaAfaAfcAfgGfuCfuagsasa | 12 |
| SNK-0109 | CfsusagacCfuGfuuuugcuuuugu | 48 | AfsCfsaAfAfAfgCfaAfaAfcAfgGfuCfuagsasa | 12 |
| SNK-0110 | csusAfgaCfuGfuuuugcuuuugu | 49 | AfsCfsaAfAfAfgCfaAfaAfcAfgGfuCfuagsasa | 12 |

In some preferred embodiments, the antisense strand includes a nucleotide sequence from positions 1 to 21 of 5′-AfsCfsaAfAfagCfaAfaAfcAfgGfuCfuagsasa-3′ (SEQ ID No. 13). When the antisense strand has the foregoing sequence, the RNAi agent has a better inhibitory effect on PCSK9.

In some preferred embodiments, the sense strand includes a nucleotide sequence of 5′-CfsusAfgacCfuGfUfUfuugcuuuugu-3′ (SEQ ID No. 4), and the antisense strand includes a nucleotide sequence of 5′-AfsCfsaAfAfagCfaAfaAfcAfgGfuCfuagsasa-3′ (SEQ ID No. 13).

In some preferred embodiments, the sense strand includes a nucleotide sequence of 5′-CfsusAfgacCfuGfUfUfuuGfcuuuugu-3′ (SEQ ID No. 5), and the antisense strand includes a nucleotide sequence of 5′-AfsCfsaAfAfagCfaAfaAfcAfgGfuCfuagsasa-3′ (SEQ ID No. 13).

In some preferred embodiments, the sense strand includes a nucleotide sequence of 5′-CfsusAfgacCfUfGfUfUfuuGfcuuuugu-3′ (SEQ ID No. 6), and the antisense strand includes a nucleotide sequence of 5′-AfsCfsaAfAfagCfaAfaAfcAfgGfuCfuagsasa-3′ (SEQ ID No. 13).

In some preferred embodiments, the sense strand includes a nucleotide sequence of 5′-CfsusAfgacCfuGfudTuugcuuuugu-3′ (SEQ ID No. 7), and the antisense strand includes a nucleotide sequence of 5′-AfsCfsaAfAfagCfaAfaAfcAfgGfuCfuagsasa-3′ (SEQ ID No. 13).

In each sequence disclosed herein, a nucleotide represented by a lowercase letter is 2′-O-methyl nucleotide; “f” indicates that the adjacent nucleotide on the left is 2′-fluoronucleotide; “dT” indicates that the nucleotide is 2′-deoxythymidine; “s” indicates that the two adjacent nucleotides on the left and right are connected via a phosphorothioate diester bond; and “Ral” represents an inverted abasic linker.

In some embodiments, when the sense strand or antisense strand of the RNAi agent and the corresponding sequence disclosed in the present disclosure have a sequence identity of less than 100%, or differ by more than one nucleotide, the sense strand or antisense strand of the nucleic acid still exhibits an inhibitory effect on PCSK9 that is similar (e.g., efficacy equivalent to 80-120%, 85-115%, or 90-110% of that of the corresponding sequence) or equivalent (e.g., efficacy equivalent to 95-105% of that of the corresponding sequence) to that of the corresponding sequence. For example, the two bases at the 3′ end of the antisense strand (e.g., the sequence as shown in SEQ ID No. 1) may be substituted with UU, CC, GG, UG, or a combination of any two nucleic acids. Another example includes mutual substitution between 2′-O-methyl nucleotide and 2′-fluoronucleotide at modification sites not specifically mentioned in the present disclosure. Such nucleic acid sequences also fall within the scope of protection of the present disclosure.

The RNAi agent according to the present disclosure can be obtained using conventional methods in the art, for example, solid-phase synthesis and liquid-phase synthesis. Commercially customized services for the solid-phase synthesis are already available, and therefore, can be obtained through commercial purchase. The modified nucleotide groups can be introduced via nucleotide monomers with corresponding modifications.

Based on the RNAi agent (siRNA) synthesized as described above, the present disclosure may further construct shRNA expression plasmids that possess the same or similar functions as the aforementioned RNAi agents. The methods for constructing such expression plasmids are well known to those skilled in the art and will not be elaborated herein.

In some embodiments, the RNAi agent further includes at least one ligand.

When applied to different targeting drug delivery systems with reference to common knowledge in the art, the sense strand and the antisense strand in the present disclosure all have better inhibitory effects. In other words, the effect advantages of the modified sequences of the present disclosure do not depend on the selection of targeting carriers. To further improve the bioavailability and therapeutic efficacy of siRNA, in the present disclosure, the targeting drug delivery system is also optimized to obtain the following technical solutions.

In some embodiments, the at least one ligand is connected to one or more nucleotides of the antisense strand or the sense strand, and the nucleotide(s) are selected from the 5′ terminal nucleotide, the 3′ terminal nucleotide, and/or any nucleotide within the strand.

In some specific embodiments, the at least one ligand is connected to the 3′ terminal nucleotide of the sense strand.

In some embodiments, the ligand is a GalNAc derivative.

In some embodiments, the ligand is one or more GalNAc derivatives connected via a single-stranded, double-stranded, or triple-stranded branched linker.

In some embodiments, the RNAi agent includes a compound having a structure as shown in the following formula I:

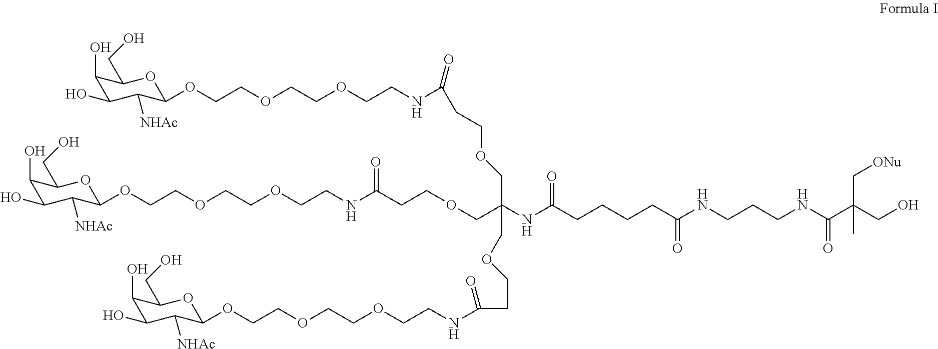

where in the formula, Nu represents a nucleic acid consisting of the sense strand and the antisense strand. This targeted drug delivery system, characterized by its structural features on the left side, enhances the cellular penetration of nucleic acid drugs (Nu), improves their intracellular stability, and can be prepared through a simple process with strong practical applicability.

### Cell and Pharmaceutical Composition

The present disclosure further provides a cell including the RNAi agent.

The cell can be used for purposes such as gene function study, disease model study, or drug screening.

In some embodiments, the cell does not develop into an animal individual. In some specific embodiments, the cell may be a microbial cell or an animal cell. However, the animal cell is not an embryonic stem cell of an animal, or a cell at various stages of formation and development (e.g., a germ cell or a fertilized egg cell).

The present disclosure further provides a pharmaceutical composition including the RNAi agent.

The pharmaceutical composition can be prepared from the RNAi agent and a pharmaceutically acceptable carrier in conventional methods. For example, the pharmaceutical composition may be an injection. The injection can be used for subcutaneous, intramuscular, or intravenous injection.

For the pharmaceutical composition according to the present disclosure, there are no special requirements for the amounts of the RNAi agent and the pharmaceutically acceptable carrier. Generally, relative to 1 part by weight of the RNAi agent, the content of the pharmaceutically acceptable carrier can be 1-100000 parts by weight (e.g., 1, 5, 10, 50, 100, 500, 1000, 5000, 10000, 50000, or 100000 parts by weight, or any value between any two of the preceding values).

For the pharmaceutical composition according to the present disclosure, the pharmaceutically acceptable carrier may be any of the various carriers conventionally used in the art, including, for example, at least one of pH buffers, protective agents, and osmotic pressure regulators. The pH buffer may be a tris(hydroxymethyl)aminomethane hydrochloride buffer with a pH value of 7.5-8.5 and/or a phosphate buffer with a pH value of 5.5-8.5, or may be preferably a phosphate buffer with a pH value of 5.5-8.5. The protective agent may be at least one of inositol, sorbitol, and sucrose. Based on the total weight of the pharmaceutical composition, the content of the protective agent may be 0.01 wt % to 30 wt % (e.g., 0.01 wt %, 0.05 wt %, 0.1 wt %, 0.5 wt %, 1 wt %, 5 wt %, 10 wt %, 15 wt %, 20 wt %, 25 wt %, or 30 wt %, or any value between any two of the preceding values). The osmotic pressure regulator may be sodium chloride and/or potassium chloride. The content of the osmotic pressure regulator should be sufficient to ensure that the osmotic pressure of the pharmaceutical composition is 200-700 milliosmoles per kilogram. Based on the required osmotic pressure, a person skilled in the art can determine the content of the osmotic pressure regulator.

Based on a preferred embodiment of the present disclosure, the pharmaceutically acceptable carrier is a liposome. The liposome can be any type of liposome capable of encapsulating nucleic acids, and may have a diameter of 25 to 1000 nm, and include, but not limited to, cholesterol and its analog or derivative.

The dosage for using the pharmaceutical composition in the present disclosure may be conventional in the art, and may be determined based on various parameters, especially the age, weight, and gender of the subject. For example, for female mice aged 3-4 months and weighing 25 g to 30 g, based on the amount of the RNAi agent in the pharmaceutical composition, the dosage of the pharmaceutical composition may be 0.01-100 mg per kg of body weight, or may be preferably 1-10 mg per kg of body weight.

### Method and Use

The present disclosure further provides a method of inhibiting expression of PCSK9 in a cell, where the method includes: contacting the cell with the RNAi agent or the pharmaceutical composition to inhibit the expression of PCSK9 in the cell.

In some embodiments, the cell is present in the body of a subject, for example, a human subject, such as a subject with a PCSK9-related disease or a subject in need of preventing the risk of a PCSK9-related disease.

In some embodiments, the cell is located in vitro. The method is intended for research or used for constructing animal models.

In some embodiments, contacting the cell with the nucleic acid inhibits the expression of PCSK9 by at least 50%, 60%, 70%, 80%, 90%, or 95% (e.g., compared to the expression level of PCSK9 before the first contact between the cell and the nucleic acid; for example, before administration of a first dose of the nucleic acid to the subject). In some embodiments, inhibiting the expression of PCSK9 reduces the level of PCSK9 protein in the serum sample of the subject by at least 50%, 60%, 70%, 80%, 90%, or 95%, compared to, for example, the expression level of PCSK9 before the first contact between the cell and the nucleic acid.

The present disclosure further provides use of the RNAi agent or the pharmaceutical composition in treatment and/or prevention of a disease associated with PCSK9. That is, a method of treating and/or preventing a disease associated with PCSK9 is provided, including: administering the RNAi agent or the pharmaceutical composition to a subject.

The present disclosure further provides use of the RNAi agent or the pharmaceutical composition in preparation of a medicament for treatment and/or prevention of a disease associated with PCSK9.

In some embodiments, the disease is: (i) a disease associated with the enhancement or elevation of PCSK9; or (ii) a disease that benefits from reduced expression of PCSK9.

In some embodiments, the disease is a cardiovascular disease. Preferably, the cardiovascular disease is selected from a group consisting of hyperlipidemia, hypercholesterolemia, coronary heart disease, myocardial infarction, stroke, and atherosclerosis.

In the present disclosure, the subject may be a mammal, including a primate (e.g., a human or a non-human primate such as a monkey or a chimpanzee), a non-primate (e.g., cattle, pig, horse, goat, rabbit, sheep, hamster, guinea pig, cat, dog, rat, or mouse), or a bird. In some embodiments, the subject is preferably a primate, and more preferably, a human.

Administration can be performed via multiple routes, depending on whether local treatment or systemic treatment is required. The dosage may refer to the foregoing description. Details are not described again herein.

In some embodiments, administration may be topical (e.g., via a transdermal patch), pulmonary (e.g., via inhalation or insufflation of powders or sprays through a nebulizer), intratracheal, intranasal, epicutaneous, transdermal, oral, or parenteral. Parenteral administration includes intravenous, intra-arterial, subcutaneous, intraperitoneal, or intramuscular injection or infusion; subcutaneous administration (e.g., via an implantation apparatus); or intracranial administration (e.g., via an intraparenchymal, intrathecal, or intraventricular route).

In some embodiments, the RNAi agent or the pharmaceutical composition is administered to a subject via subcutaneous administration, intravenous administration, and/or intramuscular administration.

### EXAMPLES

The embodiments of the present disclosure are described in detail below with reference to examples. It should be understood that these examples are only used to illustrate the present disclosure and are not intended to limit the scope of the present disclosure. For experimental methods in the following examples where specific conditions are not indicated, reference should be made to the guidance provided in the present disclosure. Alternatively, standard laboratory manuals, conventional conditions known in the art, or manufacturer-recommended procedures may be adopted.

In the following specific examples, measurement parameters related to raw material components may exhibit slight deviations within the weighing accuracy range unless otherwise specified. Parameters involving temperature and time may allow acceptable deviations resulting from instrument testing accuracy or operational precision.

### Example 1

The siRNAs listed in Table 2 were obtained through preliminary screening. To further determine the activity of these siRNAs, a dose-response experiment of the siRNAs in Table 2 was conducted using primary rhesus monkey hepatocytes. Specifically, each siRNA sample was dissolved in 100 μL of enzyme-free, sterile water to prepare a 100 μM solution. Then, 30 μL of the 100 μM test substance solution was added to 70 μL of PMonH plating medium to obtain a 30 μM solution, which was later used as a working solution for the 30 nM final concentration group. The 30 μM test substance solution was then serially diluted threefold with PMonH plating medium to generate eight concentration points, resulting in final working solution concentrations of 4.6 nM, 13.7 nM, 41.2 nM, 123.4 nM, 370.4 nM, 1111.1 nM, 3333.3 nM, and 10000 nM. Primary monkey hepatocytes were thawed from liquid nitrogen, recovered at 37° C., rinsed with serum-containing PMonH plating medium, counted, and centrifuged. After removing the supernatant, the cells were resuspended in fresh serum-containing PMonH plating medium at a concentration of 300k cells/mL. Then, 90 μL of the cell suspension was seeded into each well of a 96-well plate, resulting in 30k cells per well. The prepared sample working solutions were added to the cell suspensions to achieve final concentrations of 0.46 nM, 1.37 nM, 4.12 nM, 12.34 nM, 37.04 nM, 111.11 nM, 333.33 nM, and 1000 nM, respectively. Then, the plate was placed in an incubator with 5% carbon dioxide and cultured at a constant temperature of 37° C. for 48 hours. 48 hours later, all the medium in the 96-well culture plate was aspirated, and the plate was washed with 1×PBS buffer. Then 50 μL of the prepared Cells-to-CT lysis buffer (as per the manufacturer's recommendations) was added and mixed thoroughly. After standing for 10 minutes, 2.5 μL of stop solution was added to terminate the reaction in 2 minutes. RT-PCR was performed as per the recommendation in the High Capacity cDNA Reverse Transcription Kits (Thermo Fisher, Catalog No.: 4368814), with L of lysate per reaction. Gene expression was quantified by using real-time fluorescent PCR. The TaqMan probe for monkey PCSK9 was Mf03418189_ml, and the probe for the reference gene (monkey PPIB) was Mf02802985_ml (Thermo Fisher Scientific, Waltham, MA, USA). The PCR conditions were as follows: one cycle of 95° C. for 20 seconds, followed by 40 cycles of 95° C. for 1 second and 60° C. for 20 seconds. The real-time fluorescent PCR instrument was the QuantStudio™ 6 Pro Real-Time PCR System (Thermo Fisher). The expression of the PCSK9 gene was calculated in the 2\hat{ }-ΔΔCt method, and the expression of the monkey PPIB gene was used as internal reference. The expression level of the PCSK9 gene was expressed as a percentage relative to the control group treated with the culture medium only, and the IC₅₀ value was determined. For the results, refer to Table 3.

TABLE 2

| Compound | Sense strand | Antisense strand |
|---|---|---|
| SNK-6801 | usgsccaaAfgAfUfGfucaucaauga- | usCfsaUfuGfaugacauCfuUfuggcususu |
| | TriGalNac | |
| SNK-6802 | ascscuguUfuUfGfCfuuuuguaacu- | asGfsuUfaCfaaaagcaAfaAfcaggususu |
| | TriGalNac | |
| SNK-6803 | ususgcuuUfuGfUfAfacuugaagau- | asUfscUfuCfaaguuacAfaAfagcaasusu |
| | TriGalNac | |
| SNK-6804 | gscsuuuuGfuAfAfCfuugaagauau- | asUfsaUfcUfucaaguuAfcAfaaagcsasa |
| | TriGalNac | |
| SNK-6805 | ususuguaAfcUfUfGfaagauauuua- | usAfsaAfuAfucuucaaGfuUfacaaasasg |
| | TriGalNac | |
| SNK-6806 | csusggguUfuUfGfUfagcauuuuua- | usAfsaAfaAfugcuacaAfaAfcccagsusu |
| | TriGalNac | |
| SNK-6807 | gsusuuugUfaGfCfAfuuuuuauuaa- | usUfsaAfuAfaaaaugcUfaCfaaaacscsc |
| | TriGalNac | |
| SNK-6808 | gscsauuuUfuAfUfUfaauauaguga- | usCfsaCfuAfuauuaauAfaAfaaugcsusu |
| | TriGalNac | |
| SNK-6809 | csusagacCfuGfuuuugcuuuugu- | asCfsaAfAfAfgCfaAfaAfcAfgGfuCfuagsasa |
| | TriGalNac | |

TABLE 3

| Compound | IC₅₀, nM |
|---|---|
| SNK-6801 | No silencing effect |
| SNK-6802 | 5.10 |
| SNK-6803 | 17.50 |
| SNK-6804 | 3.80 |
| SNK-6805 | 11.87 |
| SNK-6806 | 4.21 |
| SNK-6807 | 10.76 |
| SNK-6808 | 13.88 |
| SNK-6809 | 1.10 |

The results show that SNK-6809 exhibits a significantly better inhibitory effect on the PCSK9 gene. The modification method of SNK-6809 was optimized, and the modified sequences shown in Table 4 were obtained through screening. To confirm the inhibitory effect of SNK-6809, 0.5 mL of cell culture medium (DMEM, 10% fetal bovine serum, and 1% penicillin-streptomycin solution) containing 10⁴ Hep3B cells (Procell, Cat #CL-0102) was added to the wells of a 96-well cell culture plate, and incubated overnight in a cell incubator with 5% CO₂ at 37° C. RNAiMAX (1.5 μL per well) and the small interfering nucleic acids listed in Table 4 were mixed with Opti-MEM medium, and the resulting mixture was added to the wells to achieve a final concentration of 1 nM or 10 nM in each well. The cells were then cultured for an additional 48 hours in the cell incubator with 5% CO₂ at 37° C. To extract RNA, the cell culture supernatant was aspirated completely, and the wells were rinsed with PBS. After removing residual liquid, 50 μL of prepared lysis buffer (as recommended in the Cells-to-CT Kit (Thermo Fisher Scientific, Cat #4391851c)) was added and mixed well. After 10 minutes of standing, 2.5 μL of stop solution was added to terminate the reaction for 2 minutes. RT-PCR was performed as per the recommendation in the High Capacity cDNA Reverse Transcription Kits (Thermo Fisher, Catalog No.: 4368814), with 10 μL of lysate per reaction. Gene expression was quantified by using real-time fluorescent PCR. The human PCSK9TaqMan probe was Hs00545399_ml, and the probe for the reference gene (human HPRT1) was Hs02800695_ml (Thermo Fisher Scientific, Waltham, MA, USA). The PCR conditions were as follows: one cycle of 95° C. for 20 seconds, followed by 40 cycles of 95° C. for 1 second and 60° C. for 20 seconds. The real-time fluorescent PCR instrument was the QuantStudio™ 6 Pro Real-Time PCR System (Thermo Fisher). The expression of the PCSK9 gene was calculated using the 2\hat{ }-ΔΔCt method, and the expression of the human HPRT1 gene was used as an internal reference. The expression level of the PCSK9 gene was expressed as a percentage relative to the control group treated with RNAiMAX alone. The results are shown in Table 5.

TABLE 4

| | | SEQ | | SEQ |
|---|---|---|---|---|
| | | ID | | ID |
| Compound | Sense strand (5′-3′) | No. | Antisense strand (5′-3′) | No. |
| SNK- | csusagacCfuGfuuuugcuuuug | 47 | asCfsaAfAfAfgCfaAfaAfcAfgGfuCfuags | 11 |
| 6809 | u-TriGalNac | | asa | |
| SNK- | CfsusagacCfuGfuuuugcuuuu | 48 | asCfsaAfAfAfgCfaAfaAfcAfgGfuCfuags | 11 |
| 6810 | gu-TriGalNac | | asa | |
| SNK- | csusAfgacCfuGfuuuugcuuuu | 49 | asCfsaAfAfAfgCfaAfaAfcAfgGfuCfuags | 11 |
| 6811 | gu-TriGalNac | | asa | |
| SNK- | csusagacCfuGfuuuugcuuuug | 47 | AfsCfsaAfAfAfgCfaAfaAfcAfgGfuCfua | 12 |
| 6812 | u-TriGalNac | | gsasa | |

TABLE 5

| Compound | 10 nM | 1 nM |
|---|---|---|
| SNK-6809 | 32 | 76 |
| SNK-6810 | 35 | 47 |
| SNK-6811 | 23 | 41 |
| SNK-6812 | 21 | 29 |

### Example 2

To further investigate improved modification methods, a free uptake experiment was conducted in primary human hepatocytes using siRNA conjugated with a hepatocyte-targeting ligand Tri-GalNAc (for the chemical structure, see Formula I; for the relevant sequences, see Table 6). Each siRNA-GalNAc sample was dissolved in 100 μL of enzyme-free, sterile water to obtain a 10,000 μM solution, which was then diluted to 1,000 μM and 100 μM using inVitroGRO Plating Medium. Primary human hepatocytes were retrieved from liquid nitrogen, thawed and recovered at 37° C., rinsed with serum-containing inVitroGRO Plating Medium, counted, and centrifuged. After removing the supernatant, the cells were resuspended in fresh serum-containing plating medium at a concentration of 350k cells/mL. Then, 90 μL of the cell suspension was seeded on a 96-well cell culture plate, resulting in 35k cells per well. The prepared siRNA sample working solution was added to the cell suspension to achieve final concentrations of 100 and 1000 nM. Then the plate was placed in an incubator with 5% CO₂ and cultured at a constant temperature of 37° C. for 48 hours. 48 hours later, all the medium in the 96-well culture plate was aspirated, and the plate was washed with 1×PBS buffer. RNA was then extracted in accordance with an experimental protocol of the RNeasy Mini Kit (QIAGEN). RT-PCR was performed as per the recommendation in the High Capacity cDNA Reverse Transcription Kits (Thermo Fisher, Catalog No.: 4368814), with 10 μL of lysate per reaction. Gene expression was quantified by using real-time fluorescent PCR. The TaqMan probe for human PCSK9 was Hs00545399_ml, and the probe for the reference gene (human HPRT) was Hs02800695_ml (Thermo Fisher Scientific, Waltham, MA, USA). The PCR conditions were as follows: one cycle of 95° C. for 20 seconds, followed by 40 cycles of 95° C. for 1 second and 60° C. for 20 seconds. The real-time fluorescent PCR instrument was the QuantStudio™ 6 Pro Real-Time PCR System (Thermo Fisher). The expression of the PCSK9 gene was calculated using the 2\hat{ }-ΔΔCt method, and the expression of the HPRT gene was used as an internal reference. The expression level of the PCSK9 gene under silencing was calculated and expressed as a percentage relative to the control group treated with the culture medium alone. The remaining PCSK9 mRNA expression levels are shown in Table 7.

TABLE 6

| | | SEQ | | SEQ |
|---|---|---|---|---|
| | | ID | | ID |
| Compound | Sense strand (5′-3′) | No. | Antisense strand (5′-3′) | No. |
| SNK- | CfsusAfgacCfuGfUfUfuugcuuuugu- | 4 | asCfsaAfAfAfgCfaAfaAfcAfgGfuCfuagsasa | 11 |
| 680001 | TriGalNac | | | |
| SNK- | CfsusAfgacCfuGfUfUfuuGfcuuuugu- | 5 | asCfsaAfAfAfgCfaAfaAfcAfgGfuCfuagsasa | 11 |
| 680002 | TriGalNac | | | |
| SNK- | CfsusAfgacCfUfGfUfUfuuGfcuuuugu- | 6 | asCfsaAfAfAfgCfaAfaAfcAfgGfuCfuagsasa | 11 |
| 680003 | TriGalNac | | | |
| SNK- | CfsusAfgacCfuGfudTuugcuuuugu- | 7 | asCfsaAfAfAfgCfaAfaAfcAfgGfuCfuagsasa | 11 |
| 680004 | TriGalNac | | | |
| SNK- | CfsusAfgacCfuGfudTuuGfcuuuugu- | 8 | asCfsaAfAfAfgCfaAfaAfcAfgGfuCfuagsasa | 11 |
| 680005 | TriGalNac | | | |
| SNK- | CfsusAfgacCfUfGfudTuuGfcuuuugu- | 9 | asCfsaAfAfAfgCfaAfaAfcAfgGfuCfuagsasa | 11 |
| 680006 | TriGalNac | | | |
| SNK- | CfsusAfgacCfuGfUfdTuuGfcuuuugu- | 10 | asCfsaAfAfAfgCfaAfaAfcAfgGfuCfuagsasa | 11 |
| 680007 | TriGalNac | | | |
| SNK- | CfsusAfgacCfuGfUfUfuugcuuuugu- | 4 | AfsCfsaAfAfAfgCfaAfaAfcAfgGfuCfuagsasa | 12 |
| 680008 | TriGalNac | | | |
| SNK- | CfsusAfgacCfuGfUfUfuuGfcuuuugu- | 5 | AfsCfsaAfAfAfgCfaAfaAfcAfgGfuCfuagsasa | 12 |
| 680009 | TriGalNac | | | |
| SNK- | CfsusAfgacCfUfGfUfUfuuGfcuuuugu- | 6 | AfsCfsaAfAfAfgCfaAfaAfcAfgGfuCfuagsasa | 12 |
| 680010 | TriGalNac | | | |
| SNK- | CfsusAfgacCfuGfudTuugcuuuugu- | 7 | AfsCfsaAfAfAfgCfaAfaAfcAfgGfuCfuagsasa | 12 |
| 680011 | TriGalNac | | | |
| SNK- | CfsusAfgacCfuGfudTuuGfcuuuugu- | 8 | AfsCfsaAfAfAfgCfaAfaAfcAfgGfuCfuagsasa | 12 |
| 680012 | TriGalNac | | | |
| SNK- | CfsusAfgacCfUfGfudTuuGfcuuuugu- | 9 | AfsCfsaAfAfAfgCfaAfaAfcAfgGfuCfuagsasa | 12 |
| 680013 | TriGalNac | | | |
| SNK- | CfsusAfgacCfuGfUfdTuuGfcuuuugu- | 10 | AfsCfsaAfAfAfgCfaAfaAfcAfgGfuCfuagsasa | 12 |
| 680014 | TriGalNac | | | |
| SNK- | CfsusAfgacCfuGfUfUfuugcuuuugu- | 4 | AfsCfsaAfAfagCfaAfaAfcAfgGfuCfuagsasa | 13 |
| 680016 | TriGalNac | | | |
| SNK- | CfsusAfgacCfuGfUfUfuuGfcuuuugu- | 5 | AfsCfsaAfAfagCfaAfaAfcAfgGfuCfuagsasa | 13 |
| 680017 | TriGalNac | | | |
| SNK- | CfsusAfgacCfUfGfUfUfuuGfcuuuugu- | 6 | AfsCfsaAfAfagCfaAfaAfcAfgGfuCfuagsasa | 13 |
| 680018 | TriGalNac | | | |
| SNK- | CfsusAfgacCfuGfudTuugcuuuugu- | 7 | AfsCfsaAfAfagCfaAfaAfcAfgGfuCfuagsasa | 13 |
| 680019 | TriGalNac | | | |
| SNK- | CfsusAfgacCfuGfudTuuGfcuuuugu- | 8 | AfsCfsaAfAfagCfaAfaAfcAfgGfuCfuagsasa | 13 |
| 680020 | TriGalNac | | | |
| SNK- | CfsusAfgacCfUfGfudTuuGfcuuuugu- | 9 | AfsCfsaAfAfagCfaAfaAfcAfgGfuCfuagsasa | 13 |
| 680021 | TriGalNac | | | |
| SNK- | CfsusAfgacCfuGfUfdTuuGfcuuuugu- | 10 | AfsCfsaAfAfagCfaAfaAfcAfgGfuCfuagsasa | 13 |
| 680022 | TriGalNac | | | |

TABLE 7

| Compound | 1000 nM | 100 nM |
|---|---|---|
| SNK-680001 | 47.7 | 54 |
| SNK-680002 | 47.5 | 49.5 |
| SNK-680003 | 42.1 | 56.7 |
| SNK-680004 | 68.3 | 76.2 |
| SNK-680005 | 60.6 | 61.7 |
| SNK-680006 | 74.5 | 80.3 |
| SNK-680007 | 55.4 | 59.6 |
| SNK-680008 | 38.2 | 42.3 |
| SNK-680009 | 39.7 | 42.3 |
| SNK-680010 | 38.2 | 41.7 |
| SNK-680011 | 52.2 | 61.7 |
| SNK-680012 | 48.1 | 52.4 |
| SNK-680013 | 47.4 | 54.9 |
| SNK-680014 | 42 | 44.7 |
| SNK-680016 | 34 | 34.5 |
| SNK-680017 | 33.5 | 36.5 |
| SNK-680018 | 31.2 | 36.5 |
| SNK-680019 | 39.2 | 41.5 |
| SNK-680020 | 37.3 | 41.9 |
| SNK-680021 | 57.2 | 50.3 |
| SNK-680022 | 42 | 43.8 |

It is evident that the present disclosure significantly improves the silencing effect on the PCSK9 gene through optimization of the antisense strand modifications, and further enhances the silencing effect by optimizing the sense strand modifications.

### Example 3

A free uptake experiment was conducted in primary human hepatocytes using siRNA conjugated with the hepatocyte-targeting compound Tri-GalNAc (for the chemical structure, see Formula I; for the relevant sequences, see Table 8). Each siRNA-GalNAc sample was dissolved in 100 μL of enzyme-free, sterile water to prepare a 10,000 μM solution. Then, L of the 10,000 μM test substance solution was added to 90 μL of PMonH plating medium to obtain a 1,000 μM solution, which was later used as a working solution for the 1,000 nM final concentration group. Then, the 1,000 μM test substance solution was diluted with PMonH plating medium to generate seven additional concentration points, resulting in final working solution concentrations of 0.037, 0.11, 0.33, 1.1, 3.3, 10, 100, and 1000 nM. Primary human hepatocytes were thawed from liquid nitrogen, recovered at 37° C., rinsed with serum-containing PMonH plating medium, counted, and centrifuged. After removing the supernatant, the cells were resuspended in fresh serum-containing PMonH plating medium at a concentration of 250k cells/mL. Then, 90 μL of the cell suspension was seeded into the wells of a 96-well cell culture plate, resulting in 25k cells per well. The prepared sample working solution was added to the cell suspension to achieve final concentrations of 0.037, 0.11, 0.33, 1.1, 3.3, 10, 100, and 1000 nM. Finally, the plate was placed in an incubator with 5% CO₂ and cultured at a constant temperature of 37° C. for 48 hours. 48 hours later, all the medium in the 96-well culture plate was aspirated, and the plate was washed with 1×PBS buffer. Then 50 μL of the prepared Cells-to-CT lysis buffer (as per the manufacturer's recommendations) was added and mixed thoroughly. After standing for 10 minutes, 2.5 μL of stop solution was added to terminate the reaction in 2 minutes. RT-PCR was performed as per the recommendation in the High Capacity DNA Reverse Transcription Kits (Thermo Fisher, Catalog No.: 4368814), with 10 μL of lysate in each reaction. Gene expression was quantified by using real-time fluorescent PCR. The TaqMan probe for monkey PCSK9 was Mf03418189_ml, and the probe for the reference gene (monkey PPIB) was Mf02802985_ml (Thermo Fisher Scientific, Waltham, MA, USA). The PCR conditions were as follows: one cycle of 95° C. for 20 seconds, followed by 40 cycles of 95° C. for 1 second and 60° C. for 20 seconds. The real-time fluorescent PCR instrument was the QuantStudio™ 6 Pro Real-Time PCR System (Thermo Fisher).

The expression of the PCSK9 gene was calculated using the 2\hat{ }-ΔΔCt method, and the expression of the PPIB gene was used as an internal reference. The expression level of the PCSK9 gene under silencing was calculated and expressed as a percentage relative to the control group treated with culture medium alone. The siRNA concentration required to reduce PCSK9 expression by 500 (C₅₀) in each experimental group is shown in Table 9.

TABLE 8

| | | SEQ | | SEQ |
|---|---|---|---|---|
| | | ID | | ID |
| Compound | Sense strand (5′-3′) | No. | Antisense strand (5′-3′) | No. |
| SNK- | CfsusAfgacCfuGfUfUfuugcuuuugu- | 4 | AfsCfsaAfAfagCfaAfaAfcAfgGfuCfuagsasa | 13 |
| 680016 | TriGalNac | | | |
| SNK- | CfsusAfgacCfuGfUfUfuuGfcuuuugu- | 5 | AfsCfsaAfAfagCfaAfaAfcAfgGfuCfuagsasa | 13 |
| 680017 | TriGalNac | | | |
| SNK- | CfsusAfgacCfUfGfUfUfuuGfcuuuugu- | 6 | AfsCfsaAfAfagCfaAfaAfcAfgGfuCfuagsasa | 13 |
| 680018 | TriGalNac | | | |
| SNK- | CfsusAfgacCfuGfudTuugcuuuugu- | 7 | AfsCfsaAfAfagCfaAfaAfcAfgGfuCfuagsasa | 13 |
| 680019 | TriGalNac | | | |
| SNK- | CfsusAfgacCfuGfudTuuGfcuuuugu- | 8 | AfsCfsaAfAfagCfaAfaAfcAfgGfuCfuagsasa | 13 |
| 680020 | TriGalNac | | | |
| SNK- | CfsusAfgacCfUfGfudTuuGfcuuuugu- | 9 | AfsCfsaAfAfagCfaAfaAfcAfgGfuCfuagsasa | 13 |
| 680021 | TriGalNac | | | |
| SNK- | CfsusAfgacCfuGfUfdTuuGfcuuuugu- | 10 | AfsCfsaAfAfagCfaAfaAfcAfgGfuCfuagsasa | 13 |
| 680022 | TriGalNac | | | |
| SNK- | csusagacCfuGfudTuugcuuuugu- | 14 | AfsCfsaAfAfagCfaAfaAfcAfgGfuCfuagsasa | 13 |
| 680023 | TriGalNac | | | |
| SNK- | csusagacCfuGfudTuugcuuuugu- | 14 | AfsCfsaAfAfAfgCfaAfaAfcAfgGfuCfuagsasa | 12 |
| 680015 | TriGalNac | | | |
| AD-60212 | csusagacCfuGfudTuugcuuuugu- | 14 | asCfsaAfAfAfgCfaAfaAfcAfgGfuCfuagsasa | 11 |
| | TriGalNac | | | |

TABLE 9

| | Compound | | | | |
|---|---|---|---|---|---|
| | SNK- | SNK- | SNK- | SNK- | SNK- |
| | 680016 | 680017 | 680018 | 680019 | 680020 |
| IC₅₀ (nM) | 0.610 | 0.970 | 0.956 | 2.270 | 2.190 |
| | Compound | | | | |
| | SNK- | SNK- | SNK- | SNK- | AD- |
| | 680021 | 680022 | 680023 | 680015 | 60212 |
| IC₅₀ (nM) | 2.035 | 0.994 | 1.017 | 5.599 | 25.97 |

It can be seen that the RNAi drug of the present disclosure exhibits significantly superior silencing effect on the PCSK9 gene in primary monkey hepatocytes compared to existing leading compounds. Furthermore, the optimized modification method of the present disclosure demonstrates an additional advantage in enhancing the silencing effect.

### Example 4

To further verify the inhibitory effect of siRNA on PCSK9 expression, an experiment was conducted in human PCSK9 transgenic mice. siRNA-Tri-GalNAc compound of Seq ID No. 13 in Table 8 or PBS was subcutaneously injected into the mice on Day 0. On Day 14, blood was collected to measure the PCSK9 protein level in plasma via ELISA (R&D Systems, Category No.: DPC900, United States of America). The reduction effect was expressed as a percentage relative to the PCSK9 protein level before injection. The results are shown in Table 10 below.

TABLE 10

| Compound | D 0 (D 0%) | D 14 (D 0%) |
|---|---|---|
| SNK-680016 | 100 | 48.826 |
| SNK-680017 | 100 | 46.232 |
| SNK-680018 | 100 | 42.291 |
| SNK-680019 | 100 | 45.507 |
| SNK-680020 | 100 | 62.073 |
| SNK-680021 | 100 | 62.843 |
| SNK-680022 | 100 | 65.632 |
| SNK-680023 | 100 | 56.805 |

Based on the results, the RNAi drug of the present disclosure exhibits a superior silencing effect on the PCSK9 gene in human PCSK9 transgenic mice compared to existing leading compounds. Moreover, the optimized modification method of the present disclosure provides an additional advantage in enhancing the silencing effect. Among them, SNK-680018 demonstrates significantly greater silencing effect on PCSK9.

The above examples represent only a few embodiments of the present disclosure. Although described in detail, they should not be construed as limiting the scope of the present disclosure. It should be noted that those skilled in the art may make various modifications and improvements without departing from the spirit of the present disclosure, and such variations are within the scope of protection of the present disclosure.

What is claimed is:

1. An RNAi agent comprising a sense strand and an antisense strand, wherein:
the antisense strand comprises a nucleotide sequence from positions 1 to 21 of 5′-ACAAAAGCAAAACAGGUCUAGAA-3′ (SEQ ID No. 1);
the sense strand comprises a nucleotide sequence that is at least partially complementary to the antisense strand; and
all nucleotides in the antisense strand are modified nucleotides and meet at least one of the following conditions:
i) the antisense strand does not comprise 3 consecutive identical chemical modifications at nucleotide positions 1 to 20 of the sequence as set forth in SEQ ID No. 1; or
ii) the antisense strand comprises 2′-fluoronucleotide at nucleotide position 1 of the sequence as set forth in SEQ ID No. 1.

2. The RNAi agent according to claim 1, wherein modified nucleotides at the remaining positions in the antisense strand are each independently selected from the group consisting of 2′-O-methyl nucleotide and 2′-fluoronucleotide.

3. The RNAi agent according to claim 1, wherein the antisense strand comprises 2′-fluoronucleotide at nucleotide positions 4 and 5 and 2′-O-methyl nucleotide at nucleotide position 6 of the sequence as set forth in SEQ ID No. 1.

4. The RNAi agent according to claim 1, wherein the antisense strand comprises 2′-fluoronucleotide at nucleotide positions 2 and 14 of the sequence as set forth in SEQ ID No. 1.

5. The RNAi agent according to claim 1, wherein the antisense strand comprises 2′-fluoronucleotide at nucleotide positions 2, 8, 10, 12, 14, 16, and 18 of the sequence as set forth in SEQ ID No. 1.

6. The RNAi agent according to claim 1, wherein:
the sense strand comprises a nucleotide sequence of 5′-CUAGACCUGUUUUGCUUUUGU-3′ (SEQ ID No. 2) or 5′-CUAGACCUGUTUUGCUUUUGU-3′ (SEQ ID No. 3);
all nucleotides in the sense strand are modified nucleotides;
the sense strand comprises 2′-fluoronucleotide at nucleotide positions 1 and/or 3 of the sequence as set forth in SEQ ID No. 2 or 3; and
when the sense strand comprises the nucleotide sequence as set forth in SEQ ID No. 3, the nucleotide at position 11 is 2′-deoxythymidine.

7. The RNAi agent according to claim 6, wherein modified nucleotides at the remaining positions in the sense strand are each independently selected from the group consisting of 2′-O-methyl nucleotide and 2′-fluoronucleotide.

8. The RNAi agent according to claim 6, wherein the sense strand comprises:
a nucleotide sequence of 5′-CUAGACCUGUUUUGCUUUUGU-3′ (SEQ ID No. 2), and
2′-fluoronucleotide at nucleotide position 11 of the sequence as set forth in SEQ ID No. 2.

9. The RNAi agent according to claim 6, wherein the sense strand comprises 2′-fluoronucleotide at nucleotide position 10 of the sequence as set forth in SEQ ID No. 2 or 3.

10. The RNAi agent according to claim 6, wherein the sense strand comprises 2′-fluoronucleotide at nucleotide positions 7 and/or 9 of the sequence as set forth in SEQ ID No. 2 or 3.

11. The RNAi agent according to claim 6, wherein:
nucleotides of the sense strand at nucleotide positions 8 and 14 of the sequence as set forth in SEQ ID No. 2 or 3 are each independently selected from the group consisting of 2′-fluoronucleotide and 2′-O-methyl nucleotide; and
all modified nucleotides at the remaining positions are 2′-O-methyl nucleotides.

12. The RNAi agent according to claim 1, wherein:
at least one phosphorothioate internucleotide linkage is present among the last three nucleotides at the 5′ end of the sense strand;
at least one phosphorothioate internucleotide linkage is present among the last three nucleotides at the 5′ end of the antisense strand; and
at least one phosphorothioate internucleotide linkage is present among the last five nucleotides at the 3′ end of the antisense strand.

13. The RNAi agent according to claim 1, wherein the 5′ end and/or 3′ end of the sense strand comprises an inverted abasic linker.

14. The RNAi agent according to claim 1, wherein the 3′ end of the antisense strand comprises:
i) at least one 2′-deoxythymidine; or
ii) at least one 2′-O-methyluridine.

15. The RNAi agent according to claim 1, wherein the antisense strand comprises a nucleotide sequence selected from the group consisting of SEQ ID No. 12, 13, 16, 17, 45, and 46.

16. The RNAi agent according to claim 1, wherein the sense strand comprises a nucleotide sequence selected from the group consisting of SEQ ID No. 4, 5, 6, 7, 8, 9, 10, 14, 15, 18, 19, 20, 21, 22, 23, 24, 25, 26, 27, 28, 29, 30, 31, 32, 33, 34, 35, 36, 37, 38, 39, 40, 41, 42, 43, 44, 47, 48, and 49.

17. The RNAi agent according to claim 1, wherein:
the sense strand has a sequence as set forth in SEQ ID No. 4, and the antisense strand has a sequence as set forth in SEQ ID No. 12;
the sense strand has a sequence as set forth in SEQ ID No. 5, and the antisense strand has a sequence as set forth in SEQ ID No. 12;
the sense strand has a sequence as set forth in SEQ ID No. 6, and the antisense strand has a sequence as set forth in SEQ ID No. 12;
the sense strand has a sequence as set forth in SEQ ID No. 7, and the antisense strand has a sequence as set forth in SEQ ID No. 12;
the sense strand has a sequence as set forth in SEQ ID No. 8, and the antisense strand has a sequence as set forth in SEQ ID No. 12;
the sense strand has a sequence as set forth in SEQ ID No. 9, and the antisense strand has a sequence as set forth in SEQ ID No. 12;
the sense strand has a sequence as set forth in SEQ ID No. 10, and the antisense strand has a sequence as set forth in SEQ ID No. 12;
the sense strand has a sequence as set forth in SEQ ID No. 14, and the antisense strand has a sequence as set forth in SEQ ID No. 12;
the sense strand has a sequence as set forth in SEQ ID No. 4, and the antisense strand has a sequence as set forth in SEQ ID No. 13;
the sense strand has a sequence as set forth in SEQ ID No. 5, and the antisense strand has a sequence as set forth in SEQ ID No. 13;
the sense strand has a sequence as set forth in SEQ ID No. 6, and the antisense strand has a sequence as set forth in SEQ ID No. 13;
the sense strand has a sequence as set forth in SEQ ID No. 7, and the antisense strand has a sequence as set forth in SEQ ID No. 13;
the sense strand has a sequence as set forth in SEQ ID No. 8, and the antisense strand has a sequence as set forth in SEQ ID No. 13;
the sense strand has a sequence as set forth in SEQ ID No. 9, and the antisense strand has a sequence as set forth in SEQ ID No. 13;
the sense strand has a sequence as set forth in SEQ ID No. 10, and the antisense strand has a sequence as set forth in SEQ ID No. 13;
the sense strand has a sequence as set forth in SEQ ID No. 14, and the antisense strand has a sequence as set forth in SEQ ID No. 13;
the sense strand has a sequence as set forth in SEQ ID No. 15, and the antisense strand has a sequence as set forth in SEQ ID No. 12;
the sense strand has a sequence as set forth in SEQ ID No. 4, and the antisense strand has a sequence as set forth in SEQ ID No. 16;
the sense strand has a sequence as set forth in SEQ ID No. 4, and the antisense strand has a sequence as set forth in SEQ ID No. 17;
the sense strand has a sequence as set forth in SEQ ID No. 18, and the antisense strand has a sequence as set forth in SEQ ID No. 12;
the sense strand has a sequence as set forth in SEQ ID No. 19, and the antisense strand has a sequence as set forth in SEQ ID No. 12;
the sense strand has a sequence as set forth in SEQ ID No. 20, and the antisense strand has a sequence as set forth in SEQ ID No. 12;
the sense strand has a sequence as set forth in SEQ ID No. 21, and the antisense strand has a sequence as set forth in SEQ ID No. 12;
the sense strand has a sequence as set forth in SEQ ID No. 5, and the antisense strand has a sequence as set forth in SEQ ID No. 16;
the sense strand has a sequence as set forth in SEQ ID No. 5, and the antisense strand has a sequence as set forth in SEQ ID No. 17;
the sense strand has a sequence as set forth in SEQ ID No. 22, and the antisense strand has a sequence as set forth in SEQ ID No. 12;
the sense strand has a sequence as set forth in SEQ ID No. 23, and the antisense strand has a sequence as set forth in SEQ ID No. 12;
the sense strand has a sequence as set forth in SEQ ID No. 24, and the antisense strand has a sequence as set forth in SEQ ID No. 12;
the sense strand has a sequence as set forth in SEQ ID No. 25, and the antisense strand has a sequence as set forth in SEQ ID No. 12;
the sense strand has a sequence as set forth in SEQ ID No. 6, and the antisense strand has a sequence as set forth in SEQ ID No. 16;
the sense strand has a sequence as set forth in SEQ ID No. 6, and the antisense strand has a sequence as set forth in SEQ ID No. 17;
the sense strand has a sequence as set forth in SEQ ID No. 26, and the antisense strand has a sequence as set forth in SEQ ID No. 12;
the sense strand has a sequence as set forth in SEQ ID No. 27, and the antisense strand has a sequence as set forth in SEQ ID No. 12;
the sense strand has a sequence as set forth in SEQ ID No. 28, and the antisense strand has a sequence as set forth in SEQ ID No. 12;
the sense strand has a sequence as set forth in SEQ ID No. 29, and the antisense strand has a sequence as set forth in SEQ ID No. 12;
the sense strand has a sequence as set forth in SEQ ID No. 7, and the antisense strand has a sequence as set forth in SEQ ID No. 16;
the sense strand has a sequence as set forth in SEQ ID No. 7, and the antisense strand has a sequence as set forth in SEQ ID No. 17;
the sense strand has a sequence as set forth in SEQ ID No. 30, and the antisense strand has a sequence as set forth in SEQ ID No. 12;
the sense strand has a sequence as set forth in SEQ ID No. 31, and the antisense strand has a sequence as set forth in SEQ ID No. 12;
the sense strand has a sequence as set forth in SEQ ID No. 32, and the antisense strand has a sequence as set forth in SEQ ID No. 12;
the sense strand has a sequence as set forth in SEQ ID No. 33, and the antisense strand has a sequence as set forth in SEQ ID No. 12;
the sense strand has a sequence as set forth in SEQ ID No. 8, and the antisense strand has a sequence as set forth in SEQ ID No. 16;
the sense strand has a sequence as set forth in SEQ ID No. 8, and the antisense strand has a sequence as set forth in SEQ ID No. 17;
the sense strand has a sequence as set forth in SEQ ID No. 34, and the antisense strand has a sequence as set forth in SEQ ID No. 12;
the sense strand has a sequence as set forth in SEQ ID No. 35, and the antisense strand has a sequence as set forth in SEQ ID No. 12;
the sense strand has a sequence as set forth in SEQ ID No. 36, and the antisense strand has a sequence as set forth in SEQ ID No. 12;
the sense strand has a sequence as set forth in SEQ ID No. 37, and the antisense strand has a sequence as set forth in SEQ ID No. 12;
the sense strand has a sequence as set forth in SEQ ID No. 9, and the antisense strand has a sequence as set forth in SEQ ID No. 16;
the sense strand has a sequence as set forth in SEQ ID No. 9, and the antisense strand has a sequence as set forth in SEQ ID No. 17;
the sense strand has a sequence as set forth in SEQ ID No. 38, and the antisense strand has a sequence as set forth in SEQ ID No. 12;
the sense strand has a sequence as set forth in SEQ ID No. 39, and the antisense strand has a sequence as set forth in SEQ ID No. 12;
the sense strand has a sequence as set forth in SEQ ID No. 40, and the antisense strand has a sequence as set forth in SEQ ID No. 12;
the sense strand has a sequence as set forth in SEQ ID No. 41, and the antisense strand has a sequence as set forth in SEQ ID No. 12;
the sense strand has a sequence as set forth in SEQ ID No. 10, and the antisense strand has a sequence as set forth in SEQ ID No. 16;
the sense strand has a sequence as set forth in SEQ ID No. 10, and the antisense strand has a sequence as set forth in SEQ ID No. 17;
the sense strand has a sequence as set forth in SEQ ID No. 42, and the antisense strand has a sequence as set forth in SEQ ID No. 12;
the sense strand has a sequence as set forth in SEQ ID No. 43, and the antisense strand has a sequence as set forth in SEQ ID No. 12;
the sense strand has a sequence as set forth in SEQ ID No. 44, and the antisense strand has a sequence as set forth in SEQ ID No. 12;
the sense strand has a sequence as set forth in SEQ ID No. 15, and the antisense strand has a sequence as set forth in SEQ ID No. 13;
the sense strand has a sequence as set forth in SEQ ID No. 4, and the antisense strand has a sequence as set forth in SEQ ID No. 45;
the sense strand has a sequence as set forth in SEQ ID No. 4, and the antisense strand has a sequence as set forth in SEQ ID No. 46;
the sense strand has a sequence as set forth in SEQ ID No. 18, and the antisense strand has a sequence as set forth in SEQ ID No. 13;
the sense strand has a sequence as set forth in SEQ ID No. 19, and the antisense strand has a sequence as set forth in SEQ ID No. 13;
the sense strand has a sequence as set forth in SEQ ID No. 20, and the antisense strand has a sequence as set forth in SEQ ID No. 13;
the sense strand has a sequence as set forth in SEQ ID No. 21, and the antisense strand has a sequence as set forth in SEQ ID No. 13;
the sense strand has a sequence as set forth in SEQ ID No. 5, and the antisense strand has a sequence as set forth in SEQ ID No. 45;
the sense strand has a sequence as set forth in SEQ ID No. 5, and the antisense strand has a sequence as set forth in SEQ ID No. 46;
the sense strand has a sequence as set forth in SEQ ID No. 22, and the antisense strand has a sequence as set forth in SEQ ID No. 13;
the sense strand has a sequence as set forth in SEQ ID No. 23, and the antisense strand has a sequence as set forth in SEQ ID No. 13;
the sense strand has a sequence as set forth in SEQ ID No. 24, and the antisense strand has a sequence as set forth in SEQ ID No. 13;
the sense strand has a sequence as set forth in SEQ ID No. 25, and the antisense strand has a sequence as set forth in SEQ ID No. 13;
the sense strand has a sequence as set forth in SEQ ID No. 6, and the antisense strand has a sequence as set forth in SEQ ID No. 45;
the sense strand has a sequence as set forth in SEQ ID No. 6, and the antisense strand has a sequence as set forth in SEQ ID No. 46;
the sense strand has a sequence as set forth in SEQ ID No. 26, and the antisense strand has a sequence as set forth in SEQ ID No. 13;
the sense strand has a sequence as set forth in SEQ ID No.27, and the antisense strand has a sequence as set forth in SEQ ID No. 13;
the sense strand has a sequence as set forth in SEQ ID No. 28, and the antisense strand has a sequence as set forth in SEQ ID No. 13;
the sense strand has a sequence as set forth in SEQ ID No. 29, and the antisense strand has a sequence as set forth in SEQ ID No. 13;
the sense strand has a sequence as set forth in SEQ ID No. 7, and the antisense strand has a sequence as set forth in SEQ ID No. 45;
the sense strand has a sequence as set forth in SEQ ID No. 7, and the antisense strand has a sequence as set forth in SEQ ID No. 46;
the sense strand has a sequence as set forth in SEQ ID No. 30, and the antisense strand has a sequence as set forth in SEQ ID No. 13;
the sense strand has a sequence as set forth in SEQ ID No. 31, and the antisense strand has a sequence as set forth in SEQ ID No. 13;
the sense strand has a sequence as set forth in SEQ ID No. 32, and the antisense strand has a sequence as set forth in SEQ ID No. 13;
the sense strand has a sequence as set forth in SEQ ID No. 33, and the antisense strand has a sequence as set forth in SEQ ID No. 13;
the sense strand has a sequence as set forth in SEQ ID No. 8, and the antisense strand has a sequence as set forth in SEQ ID No. 45;
the sense strand has a sequence as set forth in SEQ ID No. 8, and the antisense strand has a sequence as set forth in SEQ ID No. 46;
the sense strand has a sequence as set forth in SEQ ID No. 34, and the antisense strand has a sequence as set forth in SEQ ID No. 13;
the sense strand has a sequence as set forth in SEQ ID No. 35, and the antisense strand has a sequence as set forth in SEQ ID No. 13;
the sense strand has a sequence as set forth in SEQ ID No. 36, and the antisense strand has a sequence as set forth in SEQ ID No. 13;
the sense strand has a sequence as set forth in SEQ ID No. 37, and the antisense strand has a sequence as set forth in SEQ ID No. 13;
the sense strand has a sequence as set forth in SEQ ID No. 9, and the antisense strand has a sequence as set forth in SEQ ID No. 45;
the sense strand has a sequence as set forth in SEQ ID No. 9, and the antisense strand has a sequence as set forth in SEQ ID No. 46;
the sense strand has a sequence as set forth in SEQ ID No. 38, and the antisense strand has a sequence as set forth in SEQ ID No. 13;
the sense strand has a sequence as set forth in SEQ ID No. 39, and the antisense strand has a sequence as set forth in SEQ ID No. 13;
the sense strand has a sequence as set forth in SEQ ID No. 40, and the antisense strand has a sequence as set forth in SEQ ID No. 13;
the sense strand has a sequence as set forth in SEQ ID No. 41, and the antisense strand has a sequence as set forth in SEQ ID No. 13;
the sense strand has a sequence as set forth in SEQ ID No. 10, and the antisense strand has a sequence as set forth in SEQ ID No. 45;
the sense strand has a sequence as set forth in SEQ ID No. 10, and the antisense strand has a sequence as set forth in SEQ ID No. 46;
the sense strand has a sequence as set forth in SEQ ID No. 42, and the antisense strand has a sequence as set forth in SEQ ID No. 13;
the sense strand has a sequence as set forth in SEQ ID No. 43, and the antisense strand has a sequence as set forth in SEQ ID No. 13;
the sense strand has a sequence as set forth in SEQ ID No. 44, and the antisense strand has a sequence as set forth in SEQ ID No. 13;
the sense strand has a sequence as set forth in SEQ ID No. 47, and the antisense strand has a sequence as set forth in SEQ ID No. 12;
the sense strand has a sequence as set forth in SEQ ID No. 48, and the antisense strand has a sequence as set forth in SEQ ID No. 12; or
the sense strand has a sequence as set forth in SEQ ID No. 49, and the antisense strand has a sequence as set forth in SEQ ID No. 12.

18. The RNAi agent according to claim 1, wherein the antisense strand comprises a nucleotide sequence from positions 1 to 21 of 5′-AfsCfsaAfAfagCfaAfaAfcAfgGfuCfuagsasa-3′ (SEQ ID No. 13), and wherein:
the nucleotide represented by a lowercase letter is 2′-O-methyl nucleotide;
f indicates that an adjacent nucleotide on the left is 2′-fluoronucleotide; and
s indicates that two adjacent nucleotides on the left and right are connected by a phosphorothioate diester bond.

19. The RNAi agent according to claim 18, wherein:
the sense strand comprises a nucleotide sequence of 5′-CfsusAfgacCfuGfUfUfuugcuuuugu-3′ (SEQ ID No. 4), and the antisense strand comprises a nucleotide sequence of 5′-AfsCfsaAfAfagCfaAfaAfcAfgGfuCfuagsasa-3′ (SEQ ID No. 13);
the sense strand comprises a nucleotide sequence of 5′-CfsusAfgacCfuGfUfUfuuGfcuuuugu-3′ (SEQ ID No. 5), and the antisense strand comprises a nucleotide sequence of 5′-AfsCfsaAfAfagCfaAfaAfcAfgGfuCfuagsasa-3′ (SEQ ID No. 13);
the sense strand comprises a nucleotide sequence of 5′-CfsusAfgacCfUfGfUfUfuuGfcuuuugu-3′ (SEQ ID No. 6), and the antisense strand comprises a nucleotide sequence of 5′-AfsCfsaAfAfagCfaAfaAfcAfgGfuCfuagsasa-3′ (SEQ ID No. 13); or
the sense strand comprises a nucleotide sequence of 5′-CfsusAfgacCfuGfudTuugcuuuugu-3′ (SEQ ID No. 7), and the antisense strand comprises a nucleotide sequence of 5′-AfsCfsaAfAfagCfaAfaAfcAfgGfuCfuagsasa-3′ (SEQ ID No. 13).

20. The RNAi agent according to claim 1, further comprising at least one ligand.

21. The RNAi agent according to claim 20, wherein the at least one ligand is connected to one or more nucleotides of the antisense strand or the sense strand, and the nucleotide is selected from the group consisting of a 5′ terminal nucleotide, a 3′ terminal nucleotide, and any nucleotide within the strand.

22. The RNAi agent according to claim 20, wherein the at least one ligand is a GalNAc derivative.

23. The RNAi agent according to claim 20, wherein the at least one ligand is one or more GalNAc derivatives connected via a single-stranded, double-stranded, or triple-stranded branched linker.

24. The RNAi agent according to claim 1, wherein the RNAi agent comprises a compound having a structure as shown below: 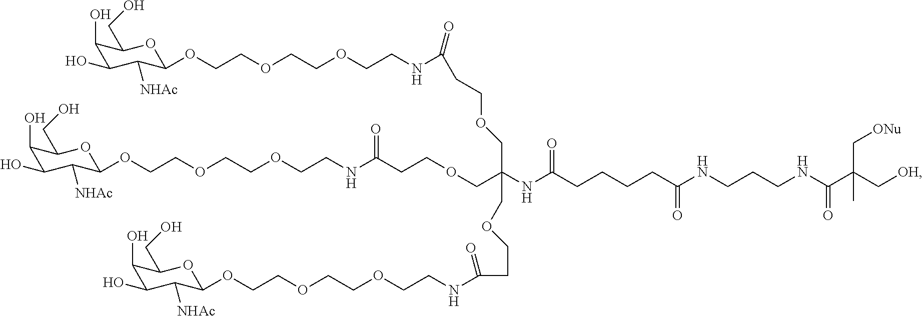
wherein Nu represents a nucleic acid consisting of the sense strand and the antisense strand.

25. A pharmaceutical composition comprising the RNAi agent according to claim 1.

26. A method of inhibiting expression of PCSK9 in a cell, the method comprising:
contacting the cell with the RNAi agent according to claim 1 or a pharmaceutical composition comprising the RNAi agent, to inhibit the expression of PCSK9 in the cell.

27. A method of treating or preventing a disease associated with PCSK9 in a subject in need thereof, the method comprising:
administering the RNAi agent according to claim 1 or a pharmaceutical composition comprising the RNAi agent to the subject.

28. The method according to claim 27, wherein the disease is selected from the group consisting of:
a disease associated with enhancement or elevation of PCSK9; and
a disease that benefits from reduced expression of PCSK9.

29. The method according to claim 27, wherein the disease is a cardiovascular disease.

30. The method according to claim 29, wherein the cardiovascular disease is selected from a group consisting of hyperlipidemia, hypercholesterolemia, coronary heart disease, myocardial infarction, stroke, and atherosclerosis.

31. The method according to claim 27, wherein the RNAi agent or the pharmaceutical composition comprising the RNAi agent is administered to the subject via subcutaneous administration, intravenous administration, and/or intramuscular administration.
